(19) United States
(12) Patent Application Publication
(10) Pub. No.: US 2018/0118801 A1
(43) Pub. Date: May 3, 2018

(54) COMPOSITIONS AND METHODS FOR DIAGNOSING AND TREATING AUTOIMMUNE DISEASES

(71) Applicant: THE REGENTS OF THE UNIVERSITY OF COLORADO, a body corporate, Denver, CO (US)

(72) Inventors: Thomas DELONG, Denver, CO (US); Kathryn HASKINS, Denver, CO (US)

(21) Appl. No.: 15/695,812

(22) Filed: Sep. 5, 2017

Related U.S. Application Data
(63) Continuation of application No. PCT/US2016/020993, filed on Mar. 4, 2016.
(60) Provisional application No. 62/128,080, filed on Mar. 4, 2015.

(51) Int. Cl. C07K 14/62; G01N 33/74; G01N 33/564; G01N 33/50

(52) U.S. Cl. CPC C07K 14/62; G01N 33/74; G01N 2800/042; G01N 33/505; G01N 33/564

(57) ABSTRACT

Disclosed are compositions and methods for detecting, isolating, and/or characterizing a T cell or autoantibody associated with type I diabetes. The composition and methods comprise the use of a hybrid insulin peptide having an N-terminal amino acid sequence taken from the human insulin peptide and a C-terminal amino acid sequence taken from a secretory granule protein that are joined through a peptide bond to form an autoimmune antigen. The detecting, isolating and characterization step further includes performing an immunoassay and/or T cell proliferation assay with the disclosed hybrid insulin peptides, where preferably, the immunoassay is an ELISPOT assay.

### CROSS REFERENCE TO RELATED APPLICATIONS

This application is a continuation of PCT Application No. PCT/US2016/020993, having an international filing date of Mar. 4, 2016, which designated the United States, which PCT application claimed the benefit of U.S. Provisional Patent Application Ser. No. 62/128,080, filed Mar. 4, 2015, both of which are incorporated herein by reference in their entirety.

### STATEMENT REGARDING FEDERALLY SPONSORED RESEARCH OR DEVELOPMENT

This invention was made with government support under grants 1 K01 DK094941 and 1R01DK081166 awarded by the National Institutes of Health. The government has certain rights in the invention.

### BACKGROUND OF THE INVENTION

Diabetes mellitus is a family of disorders characterized by chronic hyperglycemia and the development of long-term vascular complications. This family of disorders includes type 1 diabetes, type 2 diabetes, gestational diabetes, and other types of diabetes.

Diabetes is generally classified as one of two types: type 1 or type 2 diabetes. Type 2 diabetes is a non-autoimmune disease that is typically diagnosed in adults. It is a progressive disease that develops when the body does not produce sufficient insulin or fails to efficiently use the insulin it produces (a phenomenon known as insulin resistance). Patients diagnosed with type 2 diabetes are typically over age 45, overweight (BMI of 25 or higher) or obese (BMI of 30 or higher), physically inactive, have hypertension (blood pressure of 140/90 mm Hg or higher in adults), and have HDL cholesterol of 35 mg/dl or lower and/or triglyceride level of 250 mg/dl.

Type 1 diabetes (T1D), also known as juvenile diabetes or insulin-dependent diabetes mellitus, is an autoimmune disease that is typically diagnosed in children (although Adult-Onset type 1 diabetes may be present in adults). Insulin-dependent diabetes mellitus (IDDM) affects 15 million people in the United States with an estimated additional 12 million people who are currently asymptomatic, and, thus, unaware that they have this disease. Risk factors for developing type 1 diabetes include presumptive genetic factors, exposure to childhood viruses or other environmental factors, and/or the presence of other autoimmune disorders. Although the genetic factors associated with type 1 diabetes are not fully understood, risks for the development of the disease have been linked to both family history and ethnicity. For example, a child that has a parent or sibling with type 1 diabetes has a higher risk of developing T1D than a child of non-diabetic parents or with non-diabetic siblings.

T1D is caused by an autoimmune response in which the insulin producing β-cells of the pancreas (also known as islet cells) are gradually destroyed. The early stage of the disease, termed insulitis, is characterized by infiltration of leukocytes into the pancreas and is associated with both pancreatic inflammation and the release of anti β-cell antibodies. As the disease progresses, the injured tissue may also attract lymphocytes, causing yet further damage to the β-cells. Also, subsequent general activation of lymphocytes, for example in response to a viral infection, food allergy, chemical, or stress, may result in yet more islet cells being destroyed. Early stages of the disease are often overlooked or misdiagnosed as clinical symptoms of diabetes typically manifest only after about 80% of the B-cells have been destroyed. Once symptoms occur, the type-1 diabetic is normally insulin dependent for life. The dysregulation of blood-glucose levels associated with diabetes can lead to blindness, kidney failure, nerve damage and is a major contributing factor in the etiology of stroke, coronary heart disease and other blood vessel disorders. Untreated or inadequately treated T1D can result in serious complications, including nephropathy, retinopathy, cardiovascular disease, stroke, and premature death. Therefore, early detection and treatment of T1 D with a goal of consistently maintaining blood glucose at levels close to normal is important to minimize risk of serious complications.

Often, people with T1 D are asymptomatic in the early stages of the disease. Many people, particularly those without known risk factors, such as a family history of T1D, may go undiagnosed until severely high blood glucose levels have developed. Currently, commonly performed diagnostic screening for T1 D includes random blood glucose testing and hemoglobin A 1C testing, both of which are relatively insensitive and non-specific. At this time, there is no cure for T1 D.

Accordingly, there is a need in the art for new methods of diagnosing and treatment of T1D. The present invention addresses that need.

The present invention and its attributes and advantages will be further understood and appreciated with reference to the detailed description below of presently contemplated embodiments, taken in conjunction with the accompanying drawings.

### SUMMARY OF THE INVENTION

The present disclosure provides for a novel method of detecting and treating subjects with T1 D. The disclosure provides a simple yet powerful method of synthesizing diabetogenic hybrid insulin peptides to determine the presence or absence of autoantigens, autoantibodies and autoimmune T cell populations in a biological sample of interest taken from a patient having, or suspected of having T1D.

Accordingly, the present disclosure is directed to a hybrid insulin peptide comprising a first peptide having at least 90% sequence identity to at least one of SEQ ID NO: 1-86, 191-192, 196, and 221 covalently linked through a peptide bond to a second peptide having at least 90% sequence identity to at least one of SEQ ID NO: 87-175, or a truncation thereof, wherein the first peptide is N-terminal or C-terminal to the second peptide.

In some embodiments, the hybrid insulin peptide is identical to at least one of SEQ ID NO: 1-86, 191-192, 196, and 221 covalently linked through a peptide bond to the second peptide, and the second peptide being identical to at least one of SEQ ID NO: 87-175, or a truncation thereof.

In some embodiments, the hybrid insulin peptide has the first peptide positioned N-terminal to the second peptide or the first peptide is positioned C-terminal to the second peptide.

In some embodiments, the hybrid insulin peptide is antigenic for a diabetogenic CD4 T cell.

In further embodiments, the first peptide of the hybrid insulin peptide contains an amino acid sequence selected from the group consisting of SEQ ID NO: 191, 192, 196 and 221.

Also provided herein is a method for detecting, isolating or characterizing a hybrid insulin peptide comprising performing an immunoassay or a T cell proliferation assay using any one of the hybrid insulin peptides described herein.

In some embodiments, the method for detecting a hybrid insulin peptide comprises performing an immunoassay, where the immunoassay is an ELISA assay such as an ELISPOT assay.

In other embodiments of the method, the hybrid insulin peptide further comprises a hybrid insulin peptide-Major Histocompatability Complex multimer used to detect, characterize and isolate T cells.

Also provided herein is a kit for detecting a hybrid insulin peptide wherein the kit comprises, for example, any hybrid insulin peptide as disclosed herein or at least one means for detecting a hybrid insulin peptide or a combination thereof.

In some embodiments, the means for detecting a hybrid insulin peptide comprises an antibody and a detectable label. The detectable label can be a fluorophore, enzymatic label or radiolabel, or any combination thereof.

In other embodiments, a method for detecting, characterizing and isolating autoantibodies using a hybrid insulin peptide as described herein is provided.

### BRIEF DESCRIPTION OF THE DRAWINGS

FIG. 1A illustrates the IFN-gamma response of the CD4 T cell clone BDC-2.5 to antigen in chromatographic fractions of β-cell extracts (black lines). Mass spectrometric analysis of fractions reveals the presence of WE14 and insulin C-peptide in chromatographic fractions. The spectral intensities (grey lines), indicating relative peptide abundance, for insulin 1 C-peptide, but not WE14, follows the BDC-2.5 antigen distribution profile. Various insulin 1 and 2 C-peptide fragments that follow the BDC-2.5 antigen distribution profile could also be identified in antigenic fractions. FIG. 1B lists a selection of co-eluting insulin 2 C-peptide fragments:

SEQ ID NO: 212
EVEDPQVAQLELGGGPGAG,
SEQ ID NO: 213
EVEDPQVAQLELGGGPGAGD,
SEQ ID NO: 214
EVEDPQVAQLELGGGPGGADL,
SEQ ID NO: 215
EVEDPQVAQLELGGGPGAGDLQ,
SEQ ID NO: 216
EVEDPQVAQLELGGGPGAGDLQT,
SEQ ID NO: 217
EVEDPQVAQLELGGGPGAGDLQTL,
SEQ ID NO: 218
EVEDPQVAQLELGGGPGAGDLQTLA,
SEQ ID NO: 219
EVEDPQVAQLELGGGPGAGDLQTLAL,
SEQ ID NO: 220
EVEDPQVAQLELGGGPG-AGDLQTLALEVAQQ.

FIGS. 2A-2E illustrate the reaction of pathogenic T cell clones to various hybrid peptide (HIP) sequences in a synthetic peptide library. FIG. 2A is a diagram of the synthesis of HIPS by chemical activation of the C-termini of left peptides with EDC/NHS, followed by quenching of residual EDC with OTT. Addition of right peptide leads to covalent linkage of the right peptides' N-terminal amine to the activated C-terminus of the left peptide. FIGS. 2B-2E show a library of 32 HIPs that were synthesized. Left peptides are C-terminal amino acid sequences of various mouse insulin C-peptide and B-chain fragments (SEQ ID NO: 200 GDLQTL, SEQ ID NO: 201 DLQTLA, SEQ ID NO: 196 LQTLAL, SEQ ID NO: 202 QTLALE, SEQ ID NO: 203 TLALEV, SEQ ID NO: 204 HLVEAL, SEQ ID NO: 205 LVEALY, SEQ ID NO: 206 VEALYL). Right peptides include the N-terminal amino acid sequences of mouse WE14 (SEQ ID NO: 207 WSRMD), IAPP1 (SEQ ID NO: 208 TPVRS), Amylin (SEQ ID NO: 209 KCNTA) and IAPP2 (SEQ ID NO: 210 NAARD) T cell clones BDC-2.5 (FIG. 2B), BDC-10.1 (FIG. 2C), BDC-9.46 (FIG. 2D) and BDC-9.3 (FIG. 2E) were used to screen the peptide library. Positive T cell responses to individual HIPs are indicated with black squares.

FIGS. 3A-3C illustrate mass spectrometric analysis of chromatographic-cell fractions revealing the presence of HIPs. FIG. 3A is an analysis of highly enriched antigen-containing size exclusion chromatographic fractions fractionated by RP-HPLC (black line). T cell responses to individual fractions are shown for BDC-2.5 (grey line). Following the proteolytic digest with AspN, the targeted MS/MS analysis of antigen-containing fractions reveals the spectrum of the HIP that contains the C-peptide amino acid sequence SEQ ID NO: 192 DLQTLAL and the WE14 sequence SEQ ID NO: 211 WSRM (FIG. 3B). The corresponding IAPP2-HIP (SEQ ID NO: 190 DLQTLALNAAR) could also be identified in AspN digested fractions that contain the antigen recognized by BDC-9.3 (FIG. 3C).

FIGS. 4A and 4B illustrate low nanomolar concentrations of HIPs activate pathogenic T cell clones. IFN-gamma T cell responses of BDC-2.5 (FIG. 4A) and BDC-9.3 (FIG. 4B) to various peptides are shown. WE14-reactive clones (FIG. 4A) respond to high concentrations of WE14 and low nanomolar concentrations of WE14-HIP. IAPP-reactive clones respond to low nanomolar concentrations of the IAPP2-HIP, but were not responsive to IAPP2 (FIG. 4B). None of the clones respond to insulin 2 C-peptide or the insulin 2 C-peptide fragment ending with the amino acid sequence SEQ ID NO: 192 DLQTLAL. Co-incubation of the SEQ ID NO: 192 DLQTLAL-fragment with WE14 or IAPP2 did not affect T cell recognition.

FIG. 5 is a table directed to insulin fragments.

FIG. 6 is a table directed to human insulin peptide sequences.

FIGS. 7A and 7B are tables directed to human peptide sequences.

FIG. 8 is a table directed to a proposed binding register.

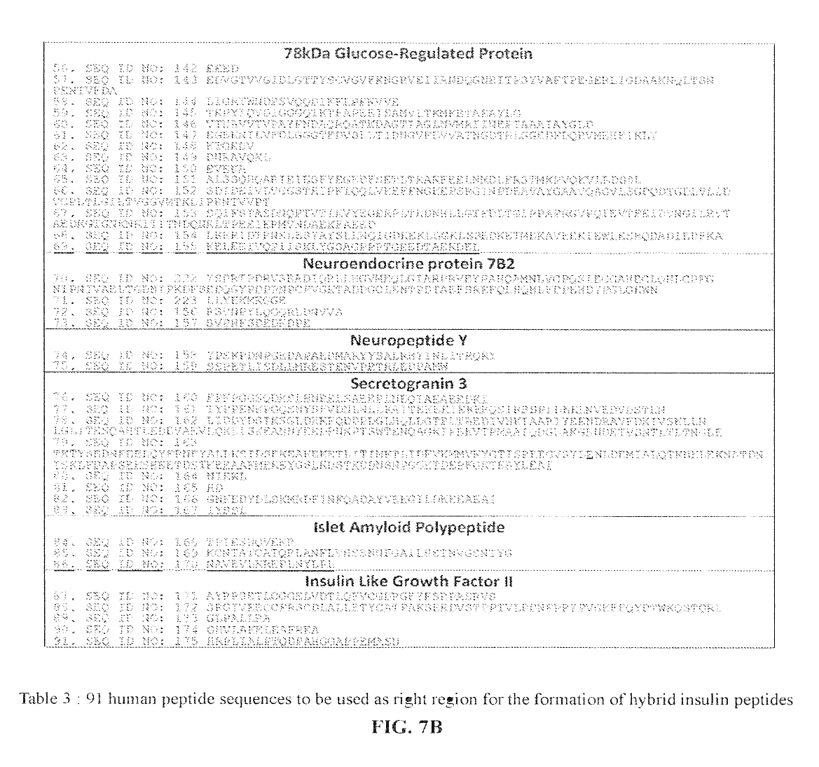

FIG. 9 is a table directed to ion observations.

FIG. 10 is another table directed to ion observations.

### DETAILED DESCRIPTION OF THE INVENTION

Insulin is a protein hormone involved in the regulation of blood sugar levels. Insulin is produced by β-cells in the islets of Langerhans of the pancreas. Insulin is produced as its precursor, preproinsulin, consisting of A and B chains of insulin linked together via a connecting C-peptide. The preproinsulin also contains a signal sequence, which is cleaved in the rough endoplasmic reticulum to produce proinsulin. Proinsulin is further processed to a mature polypeptide by various cellular endopeptidases to remove the internal C-fragment, thereby leaving the A and B chains connected via disulfide bonds. This mature insulin polypeptide is subsequently bundled into mature secretory granules until needed by the body, where it is then released into the blood stream.

T1D results in part from the autoimmune destruction of the insulin-producing β cells in the pancreas. The subsequent lack of insulin leads to increased blood and urine glucose, leading to life threatening conditions for the subject having T1D. While the cause of T1D is unknown, a process that appears to be common is an autoimmune response towards 1 cells, involving an expansion of autoreactive CD4+T helper cells and CD8+ T cells, autoantibody-producing B cells and activation of the innate immune system.

Every mammalian species that has been studied to date carries a cluster of genes coding for the so called major histocompatibility complex (MHC). This tightly linked cluster of genes code for surface antigens, which play a central role in the development of both humeral and cell-mediated immune responses. In humans, the products coded for by the MHC are referred to as Human Leukocyte Antigens or HLA.

Class I MHC molecules are 45 kD transmembrane glycoproteins, noncovalently associated with another glycoprotein, the 12 kD β-2 microglobulin. The latter is not inserted into the cell membrane, and is encoded outside the MHC Human class I molecules are of three different isotypes, termed HLA-A, -B, and -C, encoded in separate loci. The tissue expression of class I molecules is ubiquitous and codominant. MHC class I molecules present peptide antigens necessary for the activation of cytotoxic T-cells.

Class II MHC molecules are noncovalently associated heterodimers of two transmembrane glycoproteins, the 35 kD a chain and the 28 kD 3 chain. In humans, class II molecules occur as three different isotypes, termed human leukocyte antigen DR (HLA-DR), HLA-DP and HLA-DQ. Polymorphism in DR is restricted to the 1 chain, whereas both chains are polymorphic in the DP and DQ isotypes. Class II molecules are expressed co-dominantly, but in contrast to class I, exhibit a restricted tissue distribution: they are present only on the surface of cells of the immune system, for example dendritic cells, macrophages, B lymphocytes, and activated T lymphocytes. Their major biological role is to bind antigenic peptides and present them on the surface of antigen presenting cells (APC) for recognition by CD4 helper T (Th) lymphocyte. MHC class II molecules can also be expressed on the surface of non-immune system cells. For example, cells in an organ other than lymphoid cells can express MHC class II molecules during a pathological inflammatory response. These cells may include synovial cells, endothelial cells, thyroid stromal cells and glial cells.

T cells are broadly divided into cells expressing CD4 on their surface (also referred to as CD4-positive cells) and cells expressing CD8 on their surface (also referred to as CD8-positive cells). Some of the lymphocytes, referred to as B cells (or B-cells), bear on their surface a B-cell receptor. T cells can be further categorized into various populations including, but not limited to pro-inflammatory T cells, regulatory T cells, and cytotoxic T cells.

In the present context, pro-inflammatory T cells are a population of T cells capable of mediating an inflammatory reaction. Pro-inflammatory T cells generally include T helper 1 (Th1 or Type 1) and T helper 17 (Th17) subsets of T cells. These cells are also known as CD4+T cells because they express the CD4 glycoprotein on their surfaces. Th1 cells partner mainly with macrophage and can produce interferon-gamma, tumor necrosis factor-β, IL-2 and IL-10. Th1 cells promote the cellular immune response by maximizing the killing efficacy of the macrophages and the proliferation of cytotoxic CD8+ T cells. Th1 cells can also promote the production of opsonizing antibodies. T helper 17 cells (Th17) are a subset of T helper cells capable of producing interleukin 17 (IL-17) and are thought to play a key role in autoimmune diseases and in microbial infections. Th17 cells primarily produce two main members of the IL-17 family, IL-17A and IL-17F, which are involved in the recruitment, activation and migration of neutrophils. Th17 cells also secrete IL-21 and IL-22.

Regulatory T cells, also referred to as Tregs, were formerly known as suppressor T cells. Regulatory T cells are a component of the immune system that suppress immune responses of other cells. Regulatory T cells usually express CD3, CD4, CD8, CD25, and Foxp3. Additional regulatory T cell populations include Tr1, Th3, CD8+CD28-, CD69+, and Qa-1 restricted T cells. Regulatory T cells actively suppress activation of the immune system and prevent pathological self-reactivity, i.e. autoimmune disease. The critical role regulatory T cells play within the immune system is evidenced by the severe autoimmune syndrome that results from a genetic deficiency in regulatory T cells. The immunosuppressive cytokines TGF-13 and Interleukin 10 (IL-10) have also been implicated in regulatory T cell function. Similar to other T cells, a subset of regulatory T cells can develop in the thymus and this subset is usually referred to as natural Treg (or nTreg). Another type of regulatory T cell (induced Treg or iTreg) can develop in the periphery from naive CD4+ T cells. The large majority of Foxp3-expressing regulatory T cells are found within the major histocompatibility complex (MHC) class II restricted CD4-expressing (CD4+) helper T cell population and express high levels of the interleukin-2 receptor alpha chain (CD25). In addition to the Foxp3-expressing CD4+CD25+ there also appears to be a minor population of MHC class I restricted CD8+ Foxp3-expressing regulatory T cells. Unlike conventional T cells, regulatory T cells do not produce IL-2 and are therefore anergic at baseline. An alternative way of identifying regulatory T cells is to determine the DNA methylation pattern of a portion of the foxp3 gene (TSDR, Treg-specific-demethylated region) which is found demethylated in Tregs.

Efficient induction of CD4+ T cells requires that the T cells interact with antigen presenting cells (APC), i.e. cells that express MHC class II and co-stimulatory molecules. APC are dendritic cells, macrophages and activated B cells. Although nearly all nucleated cells express MHC-1, naive cytotoxic T cells (CTL) also require presentation of antigen by bone marrow-derived APC for efficient priming. Dendritic cells are highly potent inducers of CTL responses and are thought to be the principal APC involved in priming CTL's. Once primed, CTL's can recognize their cognate antigens on a wide variety of cells and respond by lysing the target cell and/or secreting cytokines, CTL's are derived from resting naive CD8 T cells and recognize antigenic peptides presented by Major Histocompatibility Complex (MHC) class I molecules. When resting CD8 T cells encounter antigenic peptides/MHC complex presented by professional antigen presenting cells, CD8 T cells will be activated and differentiated into armed CTL. Upon recognition of peptide/MHC complexes on the target cells, the antigen specific CTL will deliver a lethal hit and lyse the antigen-expressing target cells, such as virus-infected target cells or tumor cells.

The present disclosure focuses on the role of T cells in the non-obese diabetic (NOD) mouse model of autoimmune diabetes, and employs the BOC panel of pathogenic CD4 T cell clones in conjunction with proteomic analysis of -cell extracts to identify the target antigens for these clones. Recently, Applicant reported on two new autoantigens for CD4 T cells in autoimmune diabetes: chromogranin A (ChgA) and islet amyloid polypeptide (IAPP), both of which, like insulin, are -cell pro-hormonal secretory granule proteins. WE14, a naturally occurring peptide cleavage product of ChgA, was found to be antigenic in both the NOD mouse and in human patients, but because this peptide is not -cell specific and is only a weakly stimulating antigen, Applicant hypothesized that the actual ligand for the T cell clones was in some way modified. Abnormal post-translational modification (PTM) is a well-established property of many antigens in other autoimmune diseases, but with the exception of a small number of reports, modified antigens in T1D have received little attention.

As demonstrated herein, it was discovered that peptides form in pancreatic 3 cells through the formation of a peptide bond between C-termini of insulin fragments and N-termini of naturally occurring cleavage products of other β-cell secretory granule proteins such as WE14, Amylin or C-peptide. Further, it was discovered that autoreactive T cells isolated from the NOD mouse target these peptides, which are termed hybrid insulin peptides (HIPs). The demonstration of the existence of HIPs and their targeting by pathogenic T cells provides an explanation of how immune tolerance is broken in T1D. Applicant reports a completely novel PTM occurring in islet cells, namely, the formation of HIPs, which are highly antigenic for diabetogenic CD4 T cell clones.

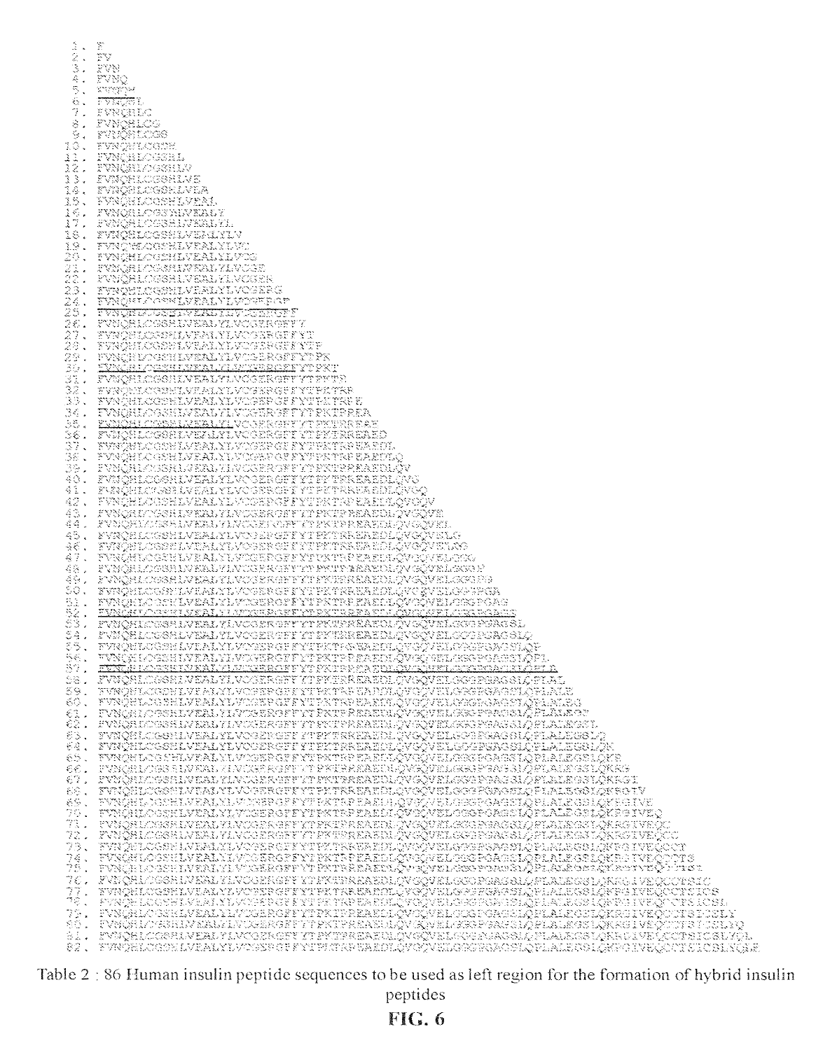

Accordingly, the present disclosure provides for an isolated hybrid insulin peptide generally having the structure A-B, where A is a “N-terminal” peptide comprising an insulin peptide sequence, and B is a “C-terminal” peptide comprising an amino acid sequence of a secretory granule protein. The A peptide may comprise any length of the insulin peptide. For instance, in some embodiments, the A peptide may be the full-length insulin peptide. In other embodiments, the A peptide may comprise, for example 1, 2, 3, 4, 5, 6, 7, 8, 9, 10, 15, 20 or more amino acids of the insulin peptide (see for instance Table 2 of FIG. 6). In essence, the A peptide may comprise any truncation of the full-length insulin peptide. In specific embodiments, the A (or “N-terminal”) peptide comprises at least 90% sequence identity, or is identical to any one of the amino acid sequences set forth in SEQ ID NO: 1-86, 191-192, 196 and 221. In other embodiments, the A peptide contains at least the amino acid sequences set forth in SEQ ID NO. 191-192, 196 and 221.

The B peptide is derived from peptide fragments of other secretory granule proteins. These secretory proteins include, but are not limited to, Insulin, Secretogranin-2, Chromogranin A, Secretogranin-1, ProSAAS, Neuroendocrine Convertase 2, 78 kDa Glucose Regulated Protein, Neuroendocrine Protein 782, Neuropeptide Y, Secretogranin-3. Islet Amyloid Polypeptide, and Insulin Like Growth Factor II.

In specific embodiments, the B peptide comprises at least 90% sequence identity, or is identical to any one of the amino acid sequence as set forth in SEQ ID NO: 87-175.

The A and B peptides are covalently linked through a peptide bond. In other embodiments, the hybrid insulin peptide may have the general structure B-A where the B peptide is now the “N-terminal” peptide and the A peptide is not the “C-terminal” peptide.

The hybrid insulin peptides may be formed using chemical synthesis methods to sequentially add amino acids to a growing chain to form the desired peptide fragment(s). In some instances, the hybrid insulin chain may be formed as a single peptide through this sequential addition. In other instances, such as when the desired peptide fragments are long, it may be beneficial to generate at least two or more peptide fragments followed by ligation of the peptide fragments together to form the hybrid peptide. One example of chemical synthesis of peptides is described in Merrifield, R. B. (1963) J. Am. Chem. Soc. 85, 2149-2154. Briefly, this method provides an amino acid corresponding to the C-terminal of the target peptide is covalently attached to an insoluble polymeric support (a “resin”). The next amino acid, with a protected a-amino acid, is activated and reacted with the resin-bound amino acid to yield an amino-protected dipeptide on the resin. Excess reactants and co-products are removed by filtration and washing. The amino-protecting group is removed and chain extension is continued with the third and subsequent protected amino acids. After the target protected peptide chain has been built up in this stepwise fashion, all side chain groups are removed and the anchoring bond between the peptide and the resin is cleaved by suitable chemical means thereby releasing the crude peptide product into solution. The desired peptide then undergoes an extensive purification procedure and is then characterized. Further examples of chemical peptide synthesis are exemplified by Houghten et al. (1980). Int. J. Pept. Protein Res., 16, 311-320; Houghten, et al. (1984), Eur. J. Biochem., 145, 157-162; Geysen et al. (1984) Proc. Natl. Acad. Sci. USA, 81, 3998-4002; Matthes, et al., (1984) The EMBO Journal, 3, 801-805, U.S. Pat. No. 6,184,344, and as described below.

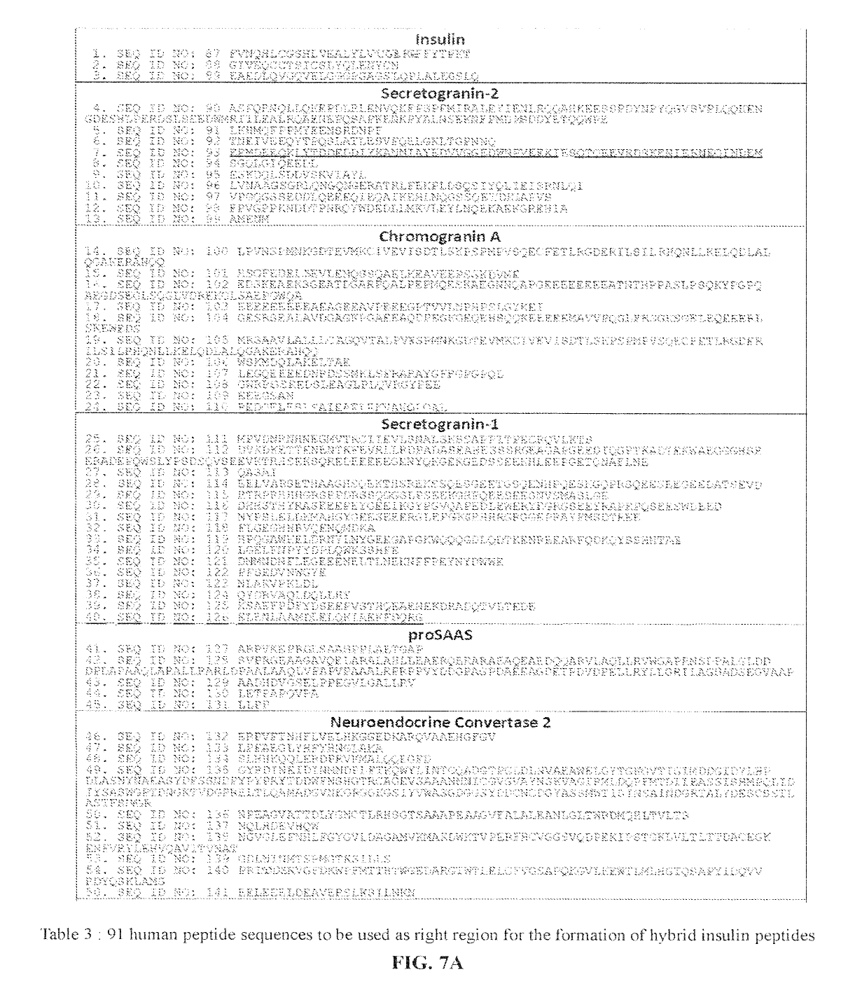

In some embodiments, it may be advantages to add short charged amino acid sequences to the peptide fragments during synthesis. Such sequences can aid in increasing solubility of a peptide or peptide fragment. An example of a short charged amino acid sequence include an “Arg-Arg-Ala” or similar motif. This motif can be added to the beginning of the N-terminal fragment and the end of the C-terminal fragment of any of the hybrid insulin peptides disclosed herein (e.g. RRA-A or B-ARR. For example, the N-terminal peptide 13 in Table 2 of FIG. 6 (SEQ ID NO: 13 FVNQHLCGSHLVE) can be extended to RRAFVNQHLCGSHLVE and the C-terminal peptide 90 in Table 3 of FIG. 7 (SEQ ID NO: 174 GHVLAKELEAFREA) can be extended to GHVLAKELEAFREAARR leads to the formation upon ligation of a hybrid peptide with the amino acid sequence RRAFVNQHLCGSHLVEGHVLAKELEAFREAARR. In more specific embodiments, an RRA motif is added to the appropriate end of the both the N-terminal and C-terminal peptides of SEQ ID NO. 1-86, 191-192, 196, 221 and SEQ ID NO: 87-175, or a truncation thereof. In some embodiments, the added motifs can be removed after formation of the complete peptide.

In other situations, it may be beneficial to create the hybrid insulin peptides using known molecular cloning techniques available to a person skilled in the art. Such techniques may include, for example the Polymerase Chain reaction to amplify pieces of nucleic acid corresponding to the A peptide and also corresponding to the B peptide using oligonucleotide forward and reverse primers that can be designed using the amino acid sequences as provided, for example in Table 2 of FIG. 6 and Table 3 of FIG. 7. The DNA sequences are subsequently ligated together to form the complete hybrid peptide sequence in a suitable cloning vector. Expression of the hybrid insulin peptide from a suitable expression vector allows for isolation or purification of the hybrid peptide (see for example, Sambrook et al. (2001), Molecular Cloning-A Laboratory Manual, Cold Spring Harbor Laboratory Press; and as disclosed in WO2013/104424, WO2001/031037, and EP0383129).

As used herein, the term “isolated” in the context of a peptide, polypeptide, fusion protein or antibody refers to a peptide, polypeptide, fusion protein or antibody which is substantially free of cellular material or contaminating proteins from the cell or tissue source from which it is derived, or substantially free of chemical precursors or other chemicals when chemically synthesized. The language “substantially free of cellular material” includes preparations of a peptide, polypeptide, fusion protein or antibody in which the peptide, polypeptide, fusion protein or antibody is separated from cellular components of the cells from which it is isolated or recombinantly produced. Thus, a peptide, polypeptide, fusion protein or antibody that is substantially free of cellular material includes preparations of a peptide, polypeptide, fusion protein or antibody having less than about 30%, 20%, 10%, or 5% (by dry weight) of heterologous protein (also referred to herein as a “contaminating protein”). When the peptide, polypeptide, fusion protein or antibody is recombinantly produced, it is also preferably substantially free of culture medium, i.e., culture medium represents less than about 20%, 10%, or 5% of the volume of the protein preparation. When the peptide, polypeptide, fusion protein or antibody is produced by chemical synthesis, it is preferably substantially free of chemical precursors or other chemicals, i.e., it is separated from chemical precursors or other chemicals which are involved in the synthesis of the peptide, polypeptide, fusion protein or antibody. Accordingly, such preparations of a peptide, polypeptide, fusion protein or antibody have less than about 30%, 20%, 10%, 5% (by dry weight) of chemical precursors or compounds other than the peptide, polypeptide, fusion protein or antibody of interest. In a preferred embodiment, a hybrid insulin peptide is isolated.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA

A “peptide” is used herein in its broadest sense to refer to a compound of two or more subunit amino acids, amino acid analogs, or other peptidomimetics. The term “peptide” thus includes short peptide sequences and also longer polypeptides and proteins.

As used herein, the term “amino acid” refers to either natural and/or unnatural or synthetic amino acids, including glycine and both the D or L optical isomers, and amino acid analogs and peptidomimetics.

Peptide “fragments” according to the invention may be made by truncation, e.g. by removal of one or more amino acids from the N and/or C-terminal ends of a polypeptide. Up to 10, up to 20, up to 30, up to 40, up to 50, up to 60, up to 75 or more amino acids may be removed from the N and/or C terminal in this way. Fragments may also be generated by one or more internal deletions.

A peptide of the invention may comprise further additional sequences. The peptide may comprise a leader sequence, i.e. a sequence at or near the amino terminus of the polypeptide that functions in targeting or regulation of the polypeptide. For example, a sequence may be included in the peptide that targets it to particular tissues in the body, or which helps the processing or folding of the peptide upon expression. Various such sequences are well known in the art and could be selected by the skilled reader depending upon, for example, the desired properties and production method of the polypeptide.

A peptide may further comprise a tag or label to identify or screen for the polypeptide, or for expression of the peptide. Suitable labels include radioisotopes such as ¹²⁵I, 32P or 35S, fluorescent labels, enzyme labels, or other protein labels such as biotin. Suitable tags may be short amino acid sequences that can be identified by routine screening methods. For example, a short amino acid sequence may be included that is recognized by a particular monoclonal antibody.

Contemplated variants of peptides containing and/or derivatives further include those containing predetermined mutations by, e.g., homologous recombination, site-directed or PCR mutagenesis, and the corresponding proteins of other animal species, including but not limited to rabbit, mouse, rat, porcine, bovine, ovine, equine and non-human primate species, and the alleles or other naturally occurring variants of the family of proteins; and derivatives wherein the protein has been covalently modified by substitution, chemical, enzymatic, or other appropriate means with a moiety other than a naturally occurring amino acid (for example a detectable moiety such as an enzyme or radioisotope).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

As used herein, “sequence identity” between two peptide sequences indicates the percentage of amino acids that are identical between the sequences. “Sequence similarity” indicates the percentage of amino acids that either are identical or that represent conservative amino acid substitutions. Preferred peptide sequences of the invention have a sequence identity of at least 60%, more preferably, at least 70% or 80%, still more preferably at least 90% and most preferably at least 95%.

A peptide of the disclosure may thus be produced from or delivered in the form of a polynucleotide which encodes, and is capable of expressing, it. Polynucleotides of the invention can be synthesized according to methods well known in the art, as described by way of example in Sambrook et al (1989, Molecular Cloning—a laboratory manual; Cold Spring Harbor Press). That is, polynucleotide sequences coding for the above-described polypeptides can be obtained using recombinant methods, such as by screening cDNA and genomic libraries, or by deriving the coding sequence for a polypeptide from a vector known to include the same. Furthermore, the desired sequences can be isolated directly from cells and tissues containing the same, using standard techniques, such as phenol extraction and PCR of cDNA or genomic DNA.

Also provided herein is a method for the diagnosis of T1D comprising the steps of providing a sample from a subject suspected of having T1 D, contacting the sample with a hybrid insulin peptide as defined herein, and determining any binding of the hybrid insulin peptide (such as by an autoantibody), thereby diagnosing a disease involving T1D. The method can be used to detect T cells or autoantibodies involved in T1D as described below. In some embodiments, the hybrid insulin peptide is attached to a solid support. In other embodiments of the method, a biological sample from a subject suspected of having T1D is contacted with an antibody raised against any hybrid insulin peptide described herein, where detection of a hybrid insulin peptide is indicative of T1D. The hybrid insulin peptide binding can be further compared to a normal control sample wherein an increase in the level of hybrid insulin peptide binding is indicative of T1 D.

Also provided herein is a method for identifying, isolating, or characterizing autoantibodies using a hybrid insulin peptide as a target epitope. In some embodiments, the method for characterizing T1D autoantibodies in a subject, comprising providing a sample from a subject suspected of having T1 D autoantibodies, detecting the presence of an autoantibody against, any one of the hybrid insulin peptides as disclosed herein, or fragment thereof in the sample wherein the presence of said autoantibody is indicative of a T1 D condition in the subject.

In other embodiments, an increased level of an autoantibody against at least one hybrid insulin peptide or fragment thereof in a sample in comparison with a normal control sample is a diagnostic indicator of T1D in a subject.

In some embodiments, the level of autoantibodies in a sample can be used to monitor treatment of T1D. Accordingly, the relative level of an autoantibody against at least one hybrid insulin peptide or fragment thereof in a sample in comparison with a sample taken before administration of a hybrid insulin peptide from the same subject is indicative of the efficacy of a therapeutic regimen. In some embodiments, the treatment regimen is inducing antigen specific immune tolerance in a subject.

As mentioned previously, autoantibodies play a role in the destruction of the Insulin producing cells, as well as other physiological conditions. Therefore, detection, characterization and isolation of the autoantibodies can be beneficial in determining the presence of T1D, as well as the specific autoantigen that may be involved in part of the autoimmune response. For instance, in some embodiments, an autoantibody is identified, isolated or characterized by obtaining a biological sample from a patient, and contacting the sample with one or more hybrid insulin peptides as disclosed herein. The sample can be blood, serum or other bodily fluid containing autoantibodies. In some embodiments, after identifying the insulin peptide(s) as the autoantigen, the samples can be tested by a focused set of hybrid peptides to further narrow the autoantigen binding site. In some situations, the identified hybrid peptide acting as an autoantigen can be administered to the patient to induce antigen specific immune tolerance. Suitable assays for characterizing autoantibodies include immunoassays such as ELISA or ELISPOT as is described in detail below.

The term “contacting” has its normal meaning and refers to combining two or more agents (e.g., polypeptides or small molecule compounds) or combining agents with cells. Contacting can occur in vitro, e.g., combining an agent with a cell or combining two cells in a test tube or other container. Contacting can also occur in vivo, e.g., by targeted delivery of an agent to a cell inside the body of a subject.

As described herein, hybrid insulin peptides may be detected by an immunoarray or similar protein array or microarray. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems. Handbook of Experimental Immunology, Vol. 1: Immunochemistry, 27.1-27.20 (1986). Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), enzyme linked immunospot assay (ELISPOT), radio immunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed hybrid insulin peptides) with an antibody to the molecule of interest or contacting an antibody to a molecule of interest (such as antibodies to the disclosed hybrid insulin peptides) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected. These methods are generally based upon the detection of a label or marker, such as any radioactive, fluorescent, biological or enzymatic tags or any other known label.

Enzyme-Linked Immunospot Assay (ELISPOT) is an immunoassay that can detect an antibody specific for a protein or antigen, as well as other molecules of interest. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, 13-galactosidase, ribonuclease, urease, catalase, malate dehydrogenase, staphylococcal nuclease, asparaginase, yeast alcohol dehydrogenase, α-glycerophosphate dehydrogenase, triose phosphate isomerase, glucose-6-phosphate dehydrogenase, glucoamylase, and acetylcholinesterase. In other embodiments, the detectable label is a fluorescent label. Various fluorescent labels include, but are not limited to, fluoresceins, rhodamines, cyanine dyes, coumarins, and the BODIPY groups of fluorescent dyes. Examples of bio luminescent detectable labels are to be found in the fluorescent reporter proteins, such as Green Fluorescent Protein (GFP) and aequorin (see, for example, U.S. Pat. Nos. 3,817,837; 3,850,752; 3,939,350; 3,996,345; 4,277,437; 4,275,149; and 4,366,241). A detectable label can further include a member of a binding pair, such as biotin/streptavidin, a metal (e.g., gold), or an epitope tag that can specifically interact with a molecule that can be detected, such as by producing a colored substrate or fluorescence. The use of fluorescent dyes is generally preferred as they can be detected at very low amounts. Furthermore, in the case where multiple antigens are reacted in an assay (or array), each antigen can be labeled with a distinct fluorescent compound for simultaneous detection. Labeled spots on the array are detected using a fluorimeter, the presence of a signal indicating an antigen bound to a specific antibody.

In some embodiments, the ELISPOT assay is performed via a nitrocellulose microtiter plate that is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined. See For example U.S. Pat. No. 8,569,074 and EP Pat. No. 1,528,395.

Enzyme-Linked Immunosorbent Assay (ELISA), or more generically termed EIA (Enzyme ImmunoAssay), can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. For descriptions of ELISA procedures, see for example, Voller, A et al., J. Clin. Pathol. 31:507-520 (1978); Butler, J. E., Meth. Enzymol. 73:482-523 (1981); “ELISA: Theory and Practice,” In: Methods in Molecule Biology, Vol. 42, Humana Press; New Jersey, 1995 and U.S. Pat. No. 4,376,110.

Variations of ELISA techniques are known to those of skill in the art. In one variation, antibodies that can bind to proteins can be immobilized onto a selected surface exhibiting protein affinity, such as a well in a polystyrene microtiter plate. Then, a test composition suspected of containing an antigen of interest can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Furthermore, numerous methods are available for immobilizing antibodies or other capture molecules to a surface. In many embodiments, the antibodies are attached to the surface through an adhesion promoting layer. There are several ways in which this layer can be formed. One way is to silanize the sensing surface to form a layer of silane molecules and another way is to use a self-assembled monolayer (SAM). There are further methods available for immobilizing capture molecules, such as chemical modification of the sensing surface (e.g. solid support, microtiter well, nanoparticle surface etc.) with reactive groups and the capture molecules with appropriate linkers, modification of the surface and capture molecules with photo reactive linkers/groups (see WO 98/27430 and WO 91/16425) immobilization via coulombic interaction (see EP0472990), or coupling via tags in chelating reactions.

In other embodiments, the hybrid insulin peptides may be used to form hybrid insulin peptide-MHC multimers for the identification, characterization and isolation of T cells through interaction with the T cell receptor.

The T cell receptor is a molecule found on the surface of T lymphocytes that is responsible for recognizing fragments of antigenic peptides, which are complexed and bound to major histocompatibility complex (MHC) molecules. A T cell receptor is a heterodimeric cell surface protein of the immunoglobulin super-family which is associated with invariant proteins of the CD3 complex involved in mediating signal transduction. The extracellular portion of native heterodimeric T cell receptor consists of two polypeptides, each of which has a membrane-proximal constant domain, and a membrane-distal variable domain. Each of the constant and variable domains includes an intra-chain disulfide bond. The variable domains contain the highly polymorphic loops analogous to the complementarity determining regions (CDRs) of antibodies.

Upon interaction of the T cell receptor with the antigen-MHC molecule (e.g. hybrid insulin peptide-MHC complex), the T cell is activated through a signal transduction cascade. The binding of the T cell receptor to the antigen-MHC molecule is known to have a low binding affinity, making it difficult for such an interaction to survive any washing attempts during purification or labeling procedures. Therefore, in some embodiments, it may be advantages to form peptide-MHC multimers to isolate and characterize T cells such that multimerization increase the amount of T cell receptor-peptide-MHC complex interaction. For example, multiple peptide-MHC complexes can be tethered together via tethering molecule such that the number of peptide-MHC complexes bound to a T cell receptor is greater than the binding of just individual peptide-MHC complexes at random. The T cell-peptide-MHC complex can be isolated or detected through well-known means such as affinity capture of flow cytometry.

In some embodiments, a peptide-MHC multimer can comprise dimers, trimers, tetramers, pentamers, hexamers, septamers and octamers or more.

In other embodiments, the peptide-MHC multimers are typically produced by biotinylating soluble MHC monomers, which can be recombinantly produced in an appropriate system. These monomers then bind to a tethering molecule, such as streptavidin or avidin, to create the multimeric structure. These tethering molecules can then be conjugated with a fluorophore or similar detectable means. The multimers with bound peptide antigen can be added to a biological sample having T cell receptor expressing cells to label and/or isolate bound T-cells via flow cytometry or similar methods (see for example Davis et al., Nat Rev Immunol. 2011 Jul. 15: 11(8): 551-558, Bakker et al., Current Opinion in Immunology, Vol. 17, No. 4 (August 2005), pp. 428-433, and Nepom et al., Journal of Immunology, Vol. 188 (2012), pp. 2477-2482, U.S. Pat. No. 8,268,964 and US Pat Pub. No. 2010/0168390).

In addition to streptavidin and avidin, other tethering molecules include: for example, IgG molecules, nucleic acid, self-assembling coiled-coil domain, dextran polymers, and streptactin. See for example European Pat. EP2361930.

In some embodiments, the peptide-MHC multimers can be used to diagnose T1D in a subject. This method for the diagnosis of T1D comprising the steps of

providing a sample from a subject suspected of having T1 D, contacting the sample with a hybrid insulin peptide-MHC multimer as disclosed herein, and determining any binding of the hybrid insulin peptide-MHC multimer complex, thereby diagnosing a disease involving T1D.

In some embodiments, a T cell proliferation assay is used comprising a hybrid insulin peptide of the disclosure to detect isolate or characterize a T cell such as is disclosed in U.S. Pat. No. 5,589,458. In other embodiments, ELISPOT assays as disclosed in Bercovici et al., Clin. Diagn. Lab Immunolv. 7(6); 2000 November and Letsch et al., Methods. Vol 31, Issue 2, October 2003, Pages 143-149, may be used to detect the secretion of various molecules of interest from a T cell. For instance, in the presence of, and subsequent recognition of a hybrid insulin peptide, a T cell population may secrete various effector molecules in response to stimulation by said hybrid peptide. A stimulated T cell may secrete, for example, tumor necrosis factor alpha, interferon gamma, interleukin-4 (IL-4), IL-5, IL-6, IL-10, IL-12, and granulocyte-macrophage colony-stimulating factor. These molecules may be detected in the ELISPOT assay and used to determine autoimmune response. Other characterization methods of T cells include flow cytometry.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey or a human), and more preferably a human.

T cells, as well as autoantibodies may be isolated/purified, for example, through the conjugation of a hybrid insulin peptide to a support structure to produce an affinity matrix, affinity column, affinity beads or the like. Activated T cells or autoantibodies can then bind to the affinity matrix, which can subsequently be removed through washing the column with an appropriate reagent, or excess free antigen (hybrid insulin peptide). See for example U.S. Pat. Nos. 7,695,713; 7,977,095; 3,639,559; WO2008/088594, and Qian et al., Biotechnol. Prag. 2009 March-April; 25(2):376-83 as disclosures relating to T cell and antibody purification and isolation.

Antibodies suitable for use with the disclosure include without limitation, polyclonal antibodies, monoclonal antibodies, chimeric antibodies, single chain antibodies, synthetic antibodies, and any antibody fragments, e.g., Fab fragments, Fab′ fragments, F(ab)2 fragments, F(ab′)2 fragments. Fd fragments. Fv fragments, dAb fragments, and isolated complementarity determining regions (“CDRs”) (see U.S. Pat. Nos. 7,037,498; 7,034,121; 7,041,870; and 7,074,405). These antibody fragments can be made by conventional procedures, such as proteolytic fragmentation procedures, as described in J. Goding, Monoclonal Antibodies: Principles and Practice, pp. 98-118 (N. Y. Academic Press 1983). Methods for preparing antibodies that are specific to a molecule of interest are well known in the art. In many embodiments, the binding affinity of an immobilized capture molecule to the respective molecule is at least 104 M⁻¹, 105 M⁻¹, 106 M⁻¹, 107 M⁻¹, 108 M⁻¹, or stronger (also see, e.g., PCT publications WO 93/17715, WO 92/08802, WO 91/00360, and WO 92/05793; Tutt, et al., J. Immunol. 147:60-69 (1991); U.S. Pat. Nos. 4,474,893, 4,714,681, 4,925,648, 5,573,920, and 5,601,819; and Kostelny et al., J. Immunol. 148:1547-1553 (1992). The antibodies used herein can be of any isotype (e.g., IgG, IgE, IgM, IgD, IgA and IgY), class (e.g., IgG1, IgG2, IgG3, IgG4, IgA1 and IgA2) or subclass of antibody molecule.

By way of example, polyclonal or monoclonal antibodies, antibody fragments, binding domains and CDRs (including engineered forms of any of the foregoing) may be created that are specific to a hybrid peptide, one or more of its respective epitopes, or conjugates of any of the foregoing, whether such antigens or epitopes are isolated from natural sources or are synthetic derivatives or variants of the natural compounds.

The term “epitope” or “antigenic determinant” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. More specifically, the antigen is a hybrid insulin peptide as described herein.

Animals may be inoculated with an antigen. Optionally, an antigen is bound or conjugated to another molecule to enhance the immune response. As used herein, a conjugate is any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

A monoclonal antibody is a single species of antibody wherein every antibody molecule recognizes the same epitope because all antibody producing cells are derived from a single B-lymphocyte cell line. The methods for generating monoclonal antibodies (MAbs) generally begin along the same lines as those for preparing polyclonal antibodies. In some embodiments, rodents such as mice and rats are used in generating monoclonal antibodies. In some embodiments, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized with a hybrid insulin peptide with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced.

Plasma B cells may be isolated from freshly prepare peripheral blood mononuclear cells of immunized animals and further selected for hybrid insulin peptide binding cells. After enrichment of antibody producing B cells, total RNA may be isolated and cDNA synthesized. DNA sequences of full length antibody or variable regions from both heavy chains and light chains may be amplified, constructed into, for example, a phage display expression vector, and transformed into E. coli. Hybrid insulin peptide specific binding full-length antibody or Fab fragments may be selected through multiple rounds of enrichment panning and then sequenced.

The present disclosure also provides pharmaceutical compositions comprising one or more of the disclosed hybrid insulin peptides together with a pharmaceutically acceptable carrier, diluent or excipient. In one embodiment, effective amounts of the pharmaceutical compositions of the disclosure are administered as a method of inducing antigen specific immune tolerance in a human type 1 diabetic subject using a hybrid insulin peptide as disclosed herein. Preferably, the composition is administered nasally or parenteral administration, i.e., intravenous, subcutaneous, intramuscular, would ordinarily be used to optimize absorption. Intravenous administration may be accomplished with the aid of an infusion pump. Furthermore, any number of hybrid peptides can be administered at a given time to induce antigen specific immune tolerance. In other embodiments, Leukocytes, and principally T cells of any type can be obtained from a subject and challenged with a hybrid insulin peptide, and then cultured in vitro using well known techniques to develop, and subsequently, the T cell population can be administered to a subject to induce antigen specific immune tolerance.

Accordingly, the compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methane sulfonate, acetate, citrate, malonate, tartrate, succinate, benzoate, ascorbate, a-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin, excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. Any material used in preparing any unit dosage form should be pharmaceutically acceptable and substantially non-toxic in the amounts employed. In addition, the active compound may be incorporated into sustained-release preparations and devices.

The liquid forms in which the present compositions may be incorporated for administration orally include aqueous solutions, suitably flavored syrups, sucrose or fructose as a sweetening agent, methyl and propyl parabens as preservatives, a dye and flavoring such as cherry or orange flavor, suspensions, and flavored emulsions with edible oils such as cottonseed oil, sesame oil, coconut oil or peanut oil, as well as elixirs and similar pharmaceutical carriers.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. Suitable dispersing or suspending agents for aqueous suspensions include synthetic and natural gums such as tragacanth, acacia, alginate, dextran, sodium carboxymethylcellulose, gelatin, methylcellulose or polyvinylpyrrolidone. Other dispersing agents which may be employed include glycerin and the like.

The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

Useful dosages of the compounds described herein can be determined by comparing their in vitro activity, and in vivo activity in animal models methods for the extrapolation of effective dosages in mice, and other animals, to humans are known to the art; for example, see U.S. Pat. No. 4,938,949. The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

The compound can be conveniently administered in a unit dosage form, for example, containing 5 to 1000 mg/m2, conveniently 10 to 750 mg/m2, most conveniently, 50 to 500 mg/m2 of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

A suitable dosage amount of the pharmaceutical composition of the present invention may vary depending on pharmaceutical formulation methods, administration methods, the patient's age, body weight, sex, pathogenic state, diet, administration time, administration route, an excretion rate and sensitivity for a used pharmaceutical composition, and physicians of ordinary skill in the art can determine an effective amount of the pharmaceutical composition for desired treatment. According to some embodiments of the disclosure, suitable dosage unit is to administer once a day with 0.001-200 mg/kg (body weight).

As used herein, “therapeutically effective amount” refers to an amount of a therapeutic agent sufficient to bring about a beneficial or desired clinical effect said dose can be administered in one or more administrations, applications, or dosages and is not intended to be limited to a particular formulation or administration route. However, the precise determination of what would be considered an effective dose may be based on factors individual to each patient, including, but not limited to, the patient's age, size, type or extent of disease, stage of the disease, route of administration, the type or extent of supplemental therapy used, ongoing disease process, and type of treatment desired (e.g., aggressive vs. conventional treatment). In further embodiments, hybrid insulin peptides may be administered to a subject to induce antigen specific immune tolerance through a delivery vehicle such as liposomes and microspheres/nanoparticles.

Liposomes are self-closed vesicular structures composed of phospholipids that entrap water in their interior. The liposomes in this invention are comprised of any bilayer forming lipid, which includes phospholipids, sphingolipids, glycosphingolipids, and ceramides. The typical size range of the liposomes is 20 nm-1000 nm. These liposomes can be rehydrated, dehydrated, partially hydrated or fully hydrated. It is also possible to employ a preliposome formulation as the liposome encapsulated biologically active material (liposome-hybrid insulin peptide complex). This formulation is composed of the biologically active material, phospholipids and cholesterol, and upon contact with water, forms liposomes. The liposomes can be mechanically stabilized using certain phospholipids, e.g. phospholipon 90H, and cholesterol at an optimum molar ratio of 2:1. The optimum ratio is expected to vary with the specific phospholipid selected. This stability can protect the liposome from GI degradation (see WO1997/031624 and U.S. Pat. No. 6,726,924).

The nanospheres/nanoparticles are typically made of a biodegradable polymer such a poly lactic acid and poly (D, L-Lactide-co-glycolide) wherein the hybrid insulin peptide coats the surface of the nanosphere, or is incorporated with in the nanosphere matrix. Nanospheres generally encompass particulate material having a dimension between about 1 nm to about 400 nm, preferably between 1 nm and 300 nm, and more preferably between 2 nm and 200 nm and most preferably from 1 nm to 100 nm. The shapes of the nanoparticles are not particularly critical: spherical nanoparticles particles are typical. See U.S. Pat. Nos. 7,943,396; 8,003,128; and US Pat. Pub. No. 2009/0202651.

Nanoparticles used with the present disclosure can comprise, for example, silicate, zinc oxide, silicon dioxide, metals, metal oxides, polymers, fullerenes or composites thereof.

As used herein, the term “pharmaceutical composition” refers to the combination of an active agent with, as desired, a carrier, inert or active, making the composition especially suitable for diagnostic or therapeutic use in vitro, in vivo, or ex vivo.

As used herein, the terms “pharmaceutically acceptable” or “pharmacologically acceptable” refer to compositions that do not substantially produce adverse reactions, e.g., toxic, allergic, or immunological reactions, when administered to a subject.

The present disclosure is also directed to a kit or system useful for practicing the methods described herein. A kit may comprise a means for detecting, isolating, and/or characterizing a hybrid insulin peptide, forming a complex of hybrid insulin peptide-MHC multimer to detect, characterize or isolate a CD4+ T cell population and to detect, characterize or isolate autoantibodies immunogenic to the hybrid insulin peptides described herein.

The kit may be a packaged combination of one or more containers, devices, or the like holding the necessary reagents, and usually including written instructions for the performance of assays. The kit may include containers to hold the materials during storage, use or both. The kit of the present invention may include any configurations and compositions for performing the various assays described herein, including, but not limited to a means of detection and a means to detect the recognition of the detection. Alternatively, a kit may only include a detection device having a means for detecting a hybrid insulin peptide or fragments thereof, and a means for recognition of the detection. Alternatively, the kit may only include a detection device having a means of detecting the hybrid insulin peptide or fragments thereof.

A means of detection may be an antibody specific to hybrid insulin peptide as disclosed herein. Alternatively, the means of detection is a substance that recognizes or detects the hybrid insulin peptide through their biological activity or structural feature. One example of biological activity is an enzymatic activity, wherein an enzyme substrate would be the recognition agent. In such case, recognition and possibly binding would lead to an observable alteration or change in the catalytic activity of said enzyme or of the enzyme substrate.

The means of detection may therefore be a protein-based, carbohydrate-based, lipid-based, natural organic-based, synthetically derived organic-based, or inorganic-based material, or any small molecule. The means of detection may also be a detection device such as, but not limited to a microfluidic device, microarray or other lateral flow devices.

In another further embodiment, the means of detection is achieved through an immune affinity procedure is any one of ELISA (e.g. ELISPOT), Western Blot, immuno-precipitation, FACS, Biochip array, Lateral Flow, Time Resolved Fluorometry, ECL procedures, or any procedure based on immune recognition.

In some embodiments, the kit may comprise a detection device having at least one compartment. A compartment may have an array of at least one means of detection wherein each means of detection is located in a defined position in the array. The term “array” as used by the methods and kits of the invention refers to an “addressed” spatial arrangement of the recognition means. Each “address” of the array is a predetermined specific spatial region containing a recognition agent. For example, an array may be a plurality of vessels (test tubes), plates, micro-wells in a micro-plate each containing a different antibody. An array may also be any solid support holding in distinct regions (dots, lines, columns) different and known recognition agents, for example antibodies. The array preferably includes built-in appropriate controls, for example, regions without the sample, regions without the antibody, regions without either, namely with solvent and reagents alone and regions containing synthetic or isolated proteins or peptides, corresponding to a positive control.

A solid support suitable for use in the kits of the present invention is typically substantially insoluble in liquid phases. Solid supports of the current invention are not limited to a specific type of support. Rather, a large number of supports are available and are known to one of ordinary skill in the art. Thus, useful solid supports include solid and semi-solid matrixes, such as aerogels and hydrogels, resins, beads, biochips (including thin film coated biochips), microfluidic chip, a silicon chip, multi-well plates (also referred to as micro-titer plates or microplates), membranes, filters, conducting and non-conducting metals, glass (including microscope slides) and magnetic supports. More specific examples of useful solid supports include silica gels, polymeric membranes, particles, derivatized plastic films, glass beads, cotton, plastic beads, alumina gels, and polysaccharides such as Sepharose, nylon, latex bead, magnetic bead, paramagnetic bead, super-paramagnetic bead, starch and the like. It should be further noted that any of the reagents included in any of the methods and kits of the invention may be provided as reagents embedded, linked, connected, attached placed or fused to any of the solid support materials described above. In some embodiments, the kit provides at least one hybrid insulin conjugated to a solid support. In other embodiments, the kit provides at least one antibody conjugated to a solid support that specifically binds to a hybrid insulin peptide.

An exemplary kit disclosed herein may contain, for example, any combination of: at least one means of detecting, characterizing or isolating a hybrid insulin peptide or fragment there of; at least one hybrid insulin peptide as disclosed herein; at least one reagent that allows the detection of an antibody-hybrid insulin peptide interaction; a detection device; a reaction compartment containing at least one means to detect hybrid insulin peptide or fragment thereof; instructions, and a control sample.

One skilled in the art may refer to general reference texts for detailed descriptions of known techniques discussed herein or equivalent techniques. These texts include Current Protocols in Molecular Biology (Ausubel et. al, eds. John Wiley & Sons, N.Y. and supplements thereto), Current Protocols in Immunology (Coligan et al, eds., John Wiley St Sons, N.Y. and supplements thereto), Current Protocols in Pharmacology (Enna et al, eds. John Wiley & Sons, N.Y. and supplements thereto) and Remington: The Science and Practice of Pharmacy (Lippincott Williams & Wilicins, 2Vt edition (2005)), for example.

Definitions of common terms in molecular biology may be found, for example, in Benjamin Lewin, Genes VII, published by Oxford University Press, 2000 (ISBN OI9879276X); Kendrew et al. (eds.); The Encyclopedia of Molecular Biology, published by Blackwell Publishers, 1994 (ISBN 0632021829); and Robert A Meyers (ed.). Molecular Biology and Biotechnology: a Comprehensive Desk Reference, published by Wiley, John & Sons, Inc., 1995 (ISBN 0471186341).

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the end-points of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### Example 1: Detection and Synthesis of Hybrid Insulin Peptides

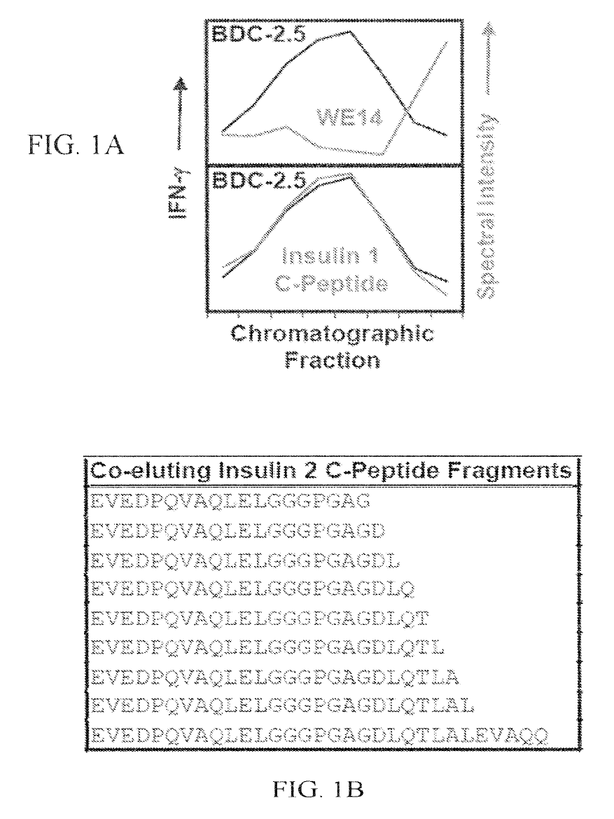

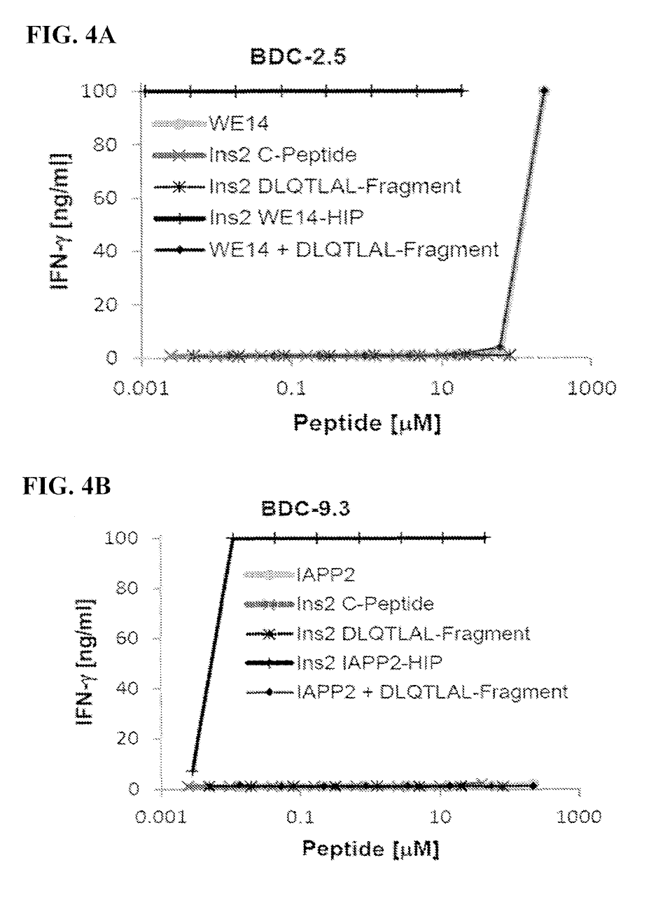

Using mass spectrometric analysis on chromatographic fractions that contain the natural 13-cell ligands for WE14-reactive T cell clones, the presence of the weakly antigenic peptide WE14 was verified. However, based on spectral intensity values that are indicative of the relative abundance of individual peptides, WE14 does not follow the chromatographic distribution profile of the natural ligand for BDC-2.5 (FIG. 1A, top). Conversely, the mouse insulin 1 C-peptide (FIG. 1A, bottom), as well as the insulin 2 C-peptide, follows the antigen distribution profile. Furthermore, a broad panel of C-peptide fragments (both insulin 1 and 2) were also identified in peak antigenic fractions (FIG. 1B) and a large number of these peptides also follows the BDC-2.5 antigen distribution profile. While these data suggest that C-peptide (and not WE14) could be the natural ligand for BDC-2.5, none of the WE14-reactive T cell clones recognize insulin C-peptide (FIG. 4A). It was therefore hypothesized that BDC-2.5 recognizes a hybrid peptide sequence in which the C-terminus of a C-peptide fragment is covalently linked to the N-terminus of the peptide WE14.

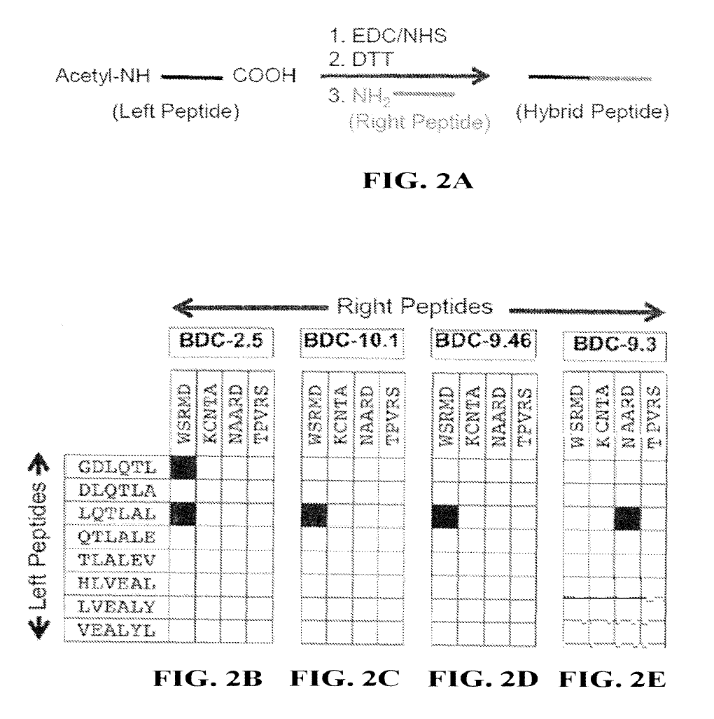

In order to test this hypothesis, a HIP peptide-screening library was synthesized using chemical crosslinking (FIG. 2A) and screened to determine whether WE14-reactive CD4 T cell clones (BDC-2.5, BDC-10.1 and BDC-9.46) isolated from different diabetic NOD mice recognize such HIP sequences. Each of the three T cell clones expresses a distinct T cell receptor (TCR). As shown in FIGS. 2B-2D, two HIP sequences that activate the WE14-reactive T cell clones from the BOC-panel were identified. Of those peptides, only one HIP sequence SEQ ID NO: 199 LQTLALWSRMD could be used to activate all three WE14-reactive clones. T cell clones BDC-9.3 and BDC-6.9, which share the same TCR, do not recognize antigen from islets of IAPP-deficient mice, indicating that IAPP is the target antigen for these clones. However, the T cell clones are not activated by IAPP peptides from overlapping peptides that span the entire pro-IAPP sequence (data not shown). A screen of the synthetic HIP library with BDC-9.3 identified a single HIP sequence (SEQ ID NO: 197 LQTLALNAARD) that is recognized by BDC-9.3 and BDC-6.9. The peptide contains the C-peptide sequence SEQ ID NO: 196 LQTLAL on the N-terminal side, and the IAPP propeptide 2 (IAPP2) sequence SEQ ID NO: 198 NAARD on the C-terminal side (FIG. 2E). The peptide IAPP2 is, like WE14, a naturally occurring cleavage product found in the secretory granules of 13-cells. It was therefore hypothesized that the ligands for the two sets of pathogenic T cell clones are HIPs containing the C-peptide fragment ending with the amino acid sequence SEQ ID NO: 192 DLQTLAL on the N-terminal side and the natural cleavage products WE14 or IAPP2 on the C-terminal side.

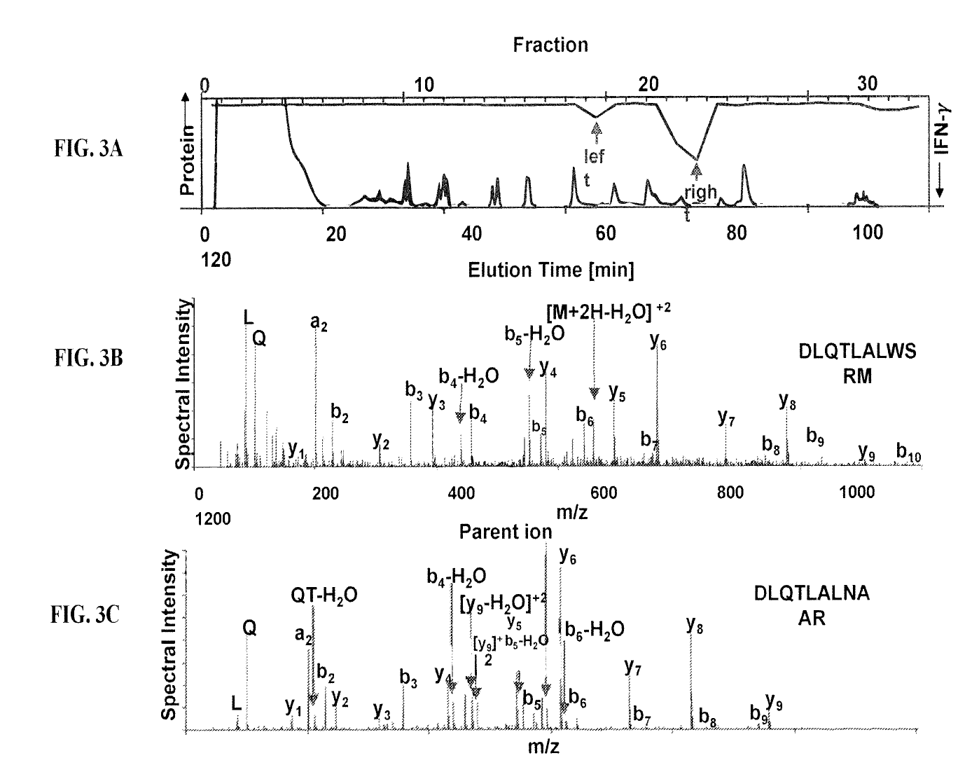

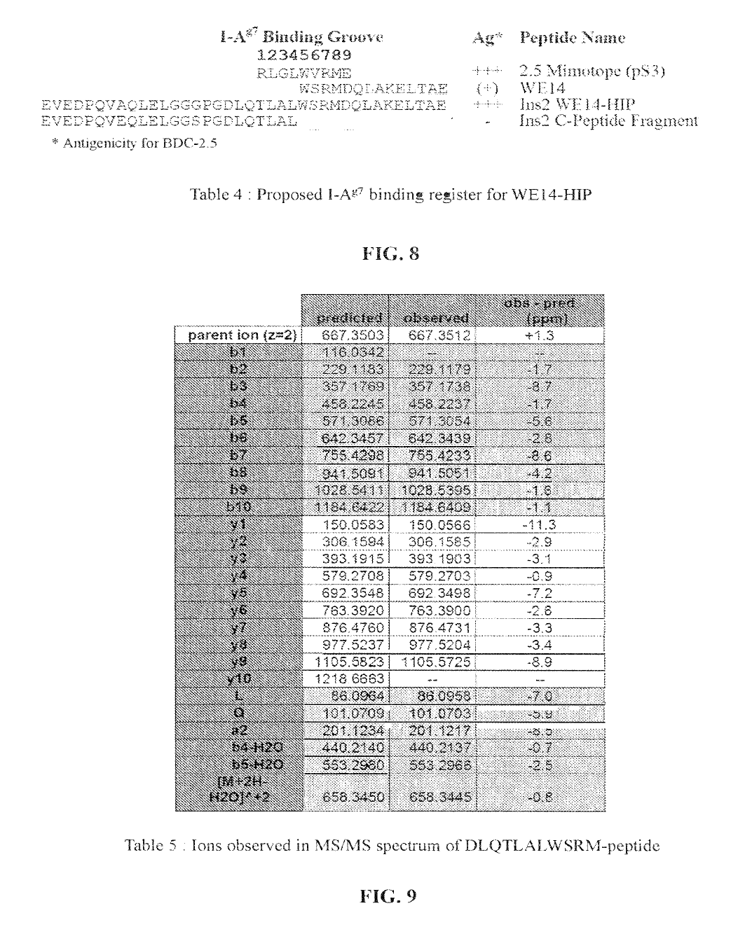

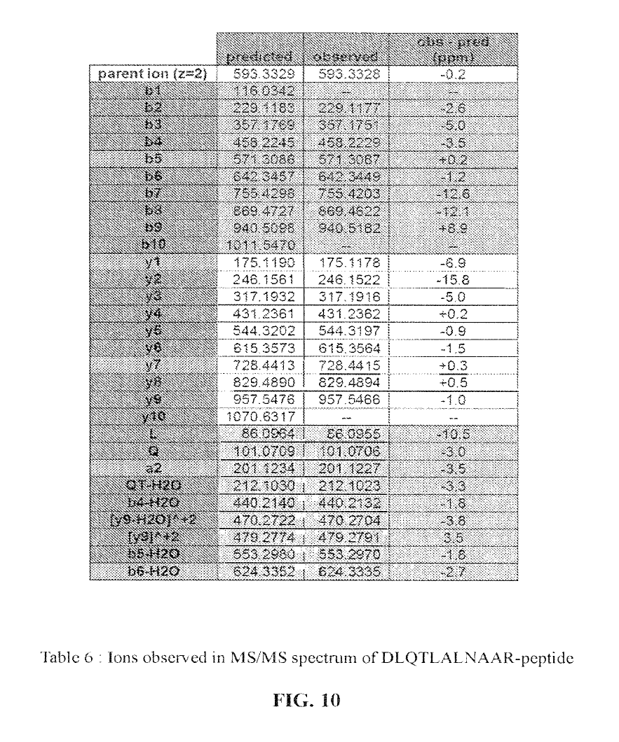

To validate the in vivo presence of HIPs in β-cell extracts, we chromatographically fractionated samples and performed mass spectrometry on antigenic (vs non-antigenic) fractions. As shown in FIG. 3A, the T cell clone BDC-2.5 responded to two chromatographic fractions indicating that at least two distinct ligands (left I right peak) exist for this T cell clone. Following the proteolytic digest with falvastacin (AspN) and MS/MS analysis of the left antigen peak (FIG. 3A), we identified the peptide SEQ ID NO: 189 DLQTLALWSRM (FIG. 3B and Table 5 of FIG. 9); this peptide spans the HIP junction recognized by all ChgA reactive T cell clones, including BDC-2.5 (compare FIG. 2B). Mice secrete two forms of insulin (Ins1/Ins2) with slightly different amino acid sequences, including differences in the C-peptide regions, and consequently, two distinct hybrid peptides can be formed between Ins1/Ins2 C-Peptide fragments (ending with the sequence SEQ ID NO: 221 DQTLAL) and WE14. However, proteolytic processing of either HIP with AspN yields the identical core peptide (SEQ ID NO: 221 DQTLAL) for both HIP, and it is therefore not possible to determine if the identified peptide originated from the Ins1 or Ins2 HIP. We have not yet identified hybrid sequences in the right antigen peak, possibly due to the low abundance of HIPs making MS-identification difficult, or alternatively the right peak may contain a secondary HIP with a different core peptide sequence. Purification of the natural ligand recognized by BDC-6.9 and BDC-9.3, followed by mass spectrometric analysis, led to the identification of the corresponding IAPP2-HIP spanning the amino acid sequence SEQ ID NO: 190 DLQTLALNMR (FIG. 3C and Table 6 of FIG. 10).

To confirm the antigenicity of the described peptides, HIPs spanning the full-length Ins2 C-Peptide fragment ending in SEQ ID NO: 196 LQTLAL on the N-terminal sides and the entire WE14 or IAPP2 sequences on the C-terminal sides were obtained. As illustrated with BDC-2.5 in FIG. 4A, the WE14-reactive T cell clones recognize the WE14-HIP at low nanomolar concentrations. As previously reported, the peptide WE14 is poorly antigenic for WE14-reactive T cell clones, requiring high peptide concentrations for T cell activation (FIG. 4A). As exemplified with BDC-9.3, the T cell clones from the second clone subset, including BDC-6.9, recognize the IAPP2-HIP at low nanomolar concentrations. Neither BDC-9.3 nor BDC-6.9 recognize the unmodified IAPP2 peptide (FIG. 4B). None of the clones respond to the full length Ins2 C-Peptide or the C-Peptide fragment ending in SEQ ID NO: 192 DLQTLAL. Furthermore, co-incubation of the C-Peptide fragment ending in SEQ ID NO: 192 DLQTLAL with unmodified WE14 or IAPP2 did not lead to an improved T cell recognition for WE14- or IAPP2-reactive T cell clones respectively, indicating that the covalent attachment of the two peptides is a prerequisite for T cell recognition (FIGS. 4A and 4B).

The role of hybrid peptides in autoimmune disease has not yet been described but could play a key role in the pathogenesis of T1D and other autoimmune diseases. The mechanism that leads to the formation of HIPs in -cells may be a side reaction of the proteolytic hydrolysis of peptide bonds in the presence of naturally occurring cleavage products such as WE14. The molecular crowding (aggregation) of peptides associated with the secretory granules of β-cells may favor this reversed proteolytic transpeptidation. This mechanism is similar to the post-translational splicing of proteins in which the two joining peptides originate from within the same protein sequence upon excision of an internal peptide fragment. As demonstrated in the experimental section, both HIPs (WE14 and IAPP2) contain the common C-peptide fragment ending with the amino acid sequence SEQ ID NO: 192 DLQTLAL, indicating that this fragment may be a preferred ligation site for the formation of HIPs. However, additional insulin ligation sites may also exist.

To date, four distinct hybrid peptide sequences, having the sequences shown below, have been identified in mouse β-cell extracts:

SEQ ID NO: 189
1. DLQTLALWSRM,
SEQ ID NO: 190
2. DLQTLALNAAR,
SEQ ID NO: 195
3. DLQTLALEVEOPQ,
SEQ ID NO: 194
4. DPQVAQLELGGEVEOPQVAQLEL.

The left region (bold) of the hybrid peptides contains the amino acid sequence of an insulin fragment, i.e. insulin peptides SEQ ID NO: 192 DLQTLAL or SEQ ID NO: 191 DPQVAQLELGG. The right region (italics) of the hybrid peptide contains an amino acid sequence of a naturally occurring cleavage product such as WE14 (WSRM), IAPP2 (NAAR) or C-Peptide (SEQ ID NO: 193 EVEDPQVAQLEL). Sequence 1 is recognized by the T cell clones BDC-2.5, BDC-10.1 and BDC-9.46. Sequence 2 is recognized by the T cell clones BDC-6.9 and BDC-9.3. Sequences 1-3 share the common insulin sequence SEQ ID NO: 192 DLQTLAL, indicating that this sequence may be a preferred ligation site for the formation of hybrid peptides. However, sequence 4 above contains a different insulin sequence (SEQ ID NO: 191 DPQVAQLELGG), indicating that other insulin peptide sequences can provide residues for the hybrid peptide formation.

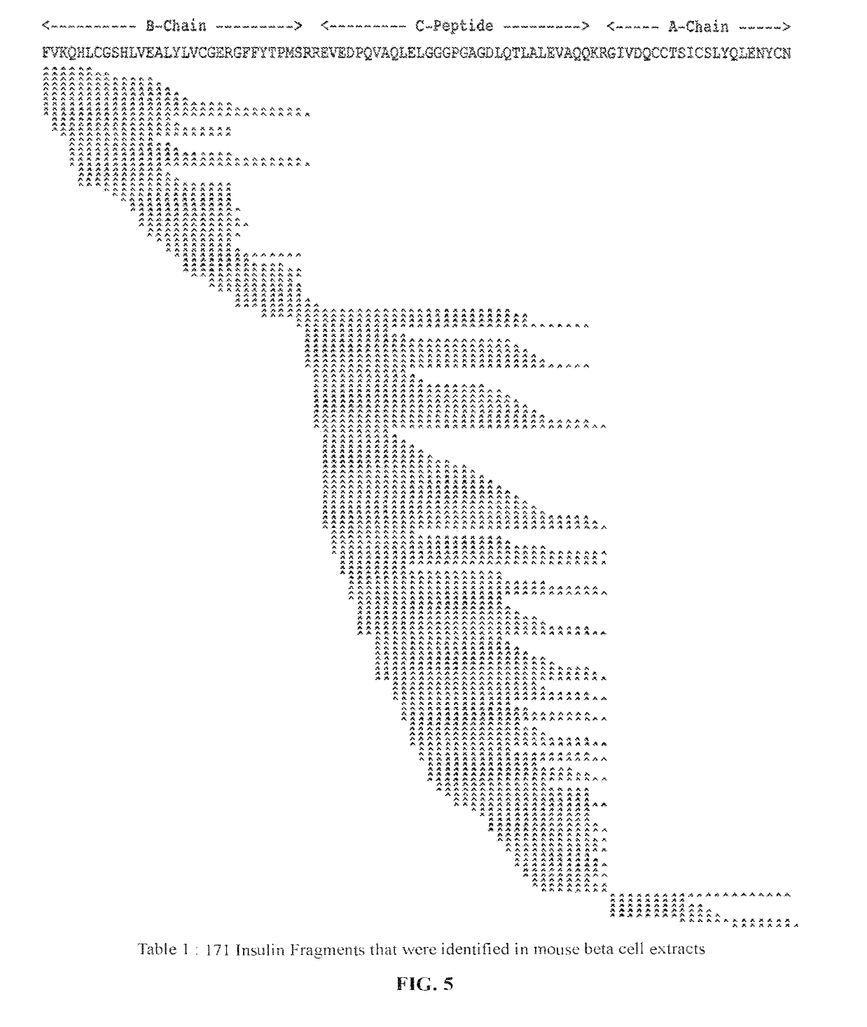

Mass spectrometric analysis of 0-cell extracts revealed the presence of 171 insulin peptides (see Table 1 of FIG. 5). Of the insulin peptides identified, several end in the amino acid sequence SEQ ID NO: 192 DLQTLAL. However, none of the identified insulin peptides end in the amino acid sequence SEQ ID NO: 191 DPQVAQLELGG, which forms the hybrid peptide of sequence 4. This demonstrates that the formation of hybrid insulin peptides in not limited to the 171 insulin peptide fragments identified in Table 1 of FIG. 5. It is envisioned that all possible proinsulin peptide fragments that can be formed can participate in the formation of a hybrid insulin peptide, and that every amino acid residue within the proinsulin sequence can provide its carboxylic acid group for the formation of hybrid insulin peptides. The corresponding full-length human insulin peptide sequences are shown in Table 2 of FIG. 6.

Mass spectrometric analysis of mouse 13 cell extracts, enriched in secretory granules, revealed the presence of the following proteins associated with the insulin secretory granules: Insulin, Secretogranin-2, Chromogranin A, Secretogranin-1, ProSAAS, Neuroendocrine Convertase 2, 78 kDa Glucose Regulated Protein, Neuroendocrine Protein 782, Neuropeptide Y, Secretogranin-3, Islet Amyloid Polypeptide, and Insulin Like Growth Factor II.

Numerous natural cleavage products of the above-listed secretory granule proteins, including, for example, the Chromogranin A peptide WE14, were identified in 13 cell extracts of NOD mice.

It is envisioned that antigenic hybrid insulin peptides are formed in humans, and that every possible peptide fragment of the secretory granule proteins can participate in formation of hybrid insulin peptides, and that every amino acid residue within the identified protein sequences can contribute its amine group to the formation of a peptide bond with an insulin fragment to form a hybrid insulin peptide. In certain embodiments, the amine groups that participate in the formation of a peptide bond to form the HIPs are contributed by the N-terminal amino acids of natural cleavage products formed by proteolytic processing of the proteins with the enzyme neuroendocrine convertase 1 or 2, which catalyze the hydrolysis of peptide bonds on the C-terminal side of two basic amino acid residues (KK, KR, RK or RR). This leads to the formation of small peptide fragments having C-terminal basic residues, which are subsequently removed by carboxypeptidases, e.g., carboxypeptidase E. The secretory granule proteins listed above contain various dibasic residues leading to the formation of 91 possible natural cleavage products that may form within the secretory granules (Table 3 of FIG. 7).

Consequently, a total of 7826 human hybrid peptide sequences can be formed by combining any one of the 86 insulin peptide sequences listed in Table 2 of FIG. 6 with any one of the 91 peptide sequences described in Table 3 of FIG. 7. For example, the formation of a peptide bond between peptide 13 in Table 2 of FIG. 6 (SEQ ID NO: 13 FVNQHLCGSHLVE) with peptide 90 in Table 3 of FIG. 7 (SEQ ID NO: 174 GHVLAKELEAFREA) leads to the formation of a hybrid peptide with the amino acid sequence SEQ ID NO: 176 FVNQHLCGSHLVEGHVLAKELEAFREA.

Conveniently, hybrid insulin peptides according to the present invention may be obtained by chemical peptide synthesis, expression in and isolation from genetically engineered microorganisms, or by purification from an individual comprising hybrid insulin peptides.

The hybrid insulin peptides or truncations of the hybrid peptides, which can be obtained through the removal of one or more N- and/or C-terminal amino acid residues, may be used as reagents in various applications. The shortest form of a truncated peptide contains at least one amino acid residue provided by each peptide. The longest form of the hybrid peptide contains the entire amino acid sequence of a peptide described in Table 2 of FIG. 6, and a peptide described in Table 3 of FIG. 7. In certain embodiments, the hybrid insulin peptides or truncations thereof are used as antigenic reagents in ELISPOT analyses and T cell proliferation assays to detect, isolate and/or characterize T cells in human subjects. In other embodiments, the hybrid insulin peptides or truncations thereof are used as reagents to make peptide-major histocompatibility complex (MHC) multimers to detect, isolate, and/or characterize T cells recognizing hybrid peptides in human subjects. In yet another embodiment, the hybrid insulin peptide sequences or truncations thereof are used as target epitopes to detect, isolate, and/or characterize autoantibodies in human subjects. In further embodiments, the hybrid insulin peptides or truncations thereof are used as reagents in strategies for the induction of antigen specific immune tolerance.

Methods

Mice: NOD and NOD RIPTAg mice were bred and maintained in the Biological Resource Center at National Jewish Health (NJH), Denver Colo. All experimental procedures were in accordance with Institutional Animal Care and Use Committee guidelines and approved by the NJH Animal Care and Use Committee.

Assays for Antigen

The antigenicity of biochemical fractions, peptides, or reaction mixtures was assessed through the IFN-y responses of T-cell clones. In 96-well microtiter plates, assay cultures contained 2×104 responder T cells, 2.5×104 NOD peritoneal exudate cells as antigen-presenting cells, and -cell antigen. ELISA (BD Biosciences) was used to determine IFN-gamma production by the responder T cells. Aliquots of cell extracts (β-Mem) were used as positive controls throughout the experiments. Test wells contained biochemical fractions, peptides, or reaction mixtures. Synthetic peptides (>95%) were obtained from CHI Scientific.

Antigen Purification

Natural antigens were enriched from 13-cell tumors of NOD RIPTAg mice through differential centrifugation followed by size exclusion chromatography. Subsequently, a final concentration of 2.7% acetonitrile and 1% trifluoracetic acid (TFA) was added to peak antigenic fractions as determined through T cell antigen assays. A total of 900 ml of this mixture was then applied to a reversed-phase high-performance liquid chromatography (RP-HPLC) Extend C18 RRHD 1.8 mm 2.1×150 mm column (Agilent). A water/acetonitrile buffer gradient (0.1% TFA) was used to elute proteins from the column, and a total of 36 fractions was collected between 0 and 120 min at a flow rate of 0.2 ml/min and a constant column temperature of 40° C. Solvents were removed from fractions through vacuum evaporation prior to T cell antigen assays and mass spectrometric analysis.

Synthesis and Testing of HIP Library

For the synthesis of a HIP peptide through chemical crosslinking of an N-terminally acetylated “left peptide” and an unmodified “right peptide”, a two-step crosslinking procedure was adopted. To ensure water solubility of peptides, “left peptides” (>85%, CHI-Scientific) were N-terminally extended and “right peptides” (>85%, CHI-Scientific) were C-terminally extended through the addition of two arginine residues separated by an alanine residue from the core amino acid sequences. A total of 10 μl “left peptide” (10 mM: SEQ ID NO: 177 Acetyl-RRAHLVEAL. SEQ ID NO: 178 Acetyl-RRALVEALY, SEQ ID NO: 179 Acetyl-RRAVEALYL, SEQ ID NO. 180 Acetyl-RRAGDLQTL, SEQ ID NO: 181 Acetyl-RRADLQTLA, SEQ ID NO: 182 Acetyl-RRALQTLAL, SEQ ID NO: 183 Acetyl-RRAQTLALE, or SEQ ID NO: 184 Acetyl-RRATLALEV) was added to 74.5 μl reaction buffer (20 mM MES, 150 mM NaCl) in a 1.5 ml eppendorf tube, followed by the addition of a carbodiimide, such as, for example, 1.5 μl freshly prepared 500 mM 1-Ethyl-3-[3-dimethylaminopropyl]carbodiimide hydrochloride (EDC) and 1.5 μl 1000 mM N-hydroxysuccinimide (NHS). After 15 min of incubation at room temperature, a reducing agent was added, for example, 2.5 ml of DTT (1000 mM) were added to quench residual EDC. Following another 15 min of incubation at room temperature, 10 ml of “right peptide” (10 mM: SEQ ID NO: 185 KCNTATARR, SEQ ID NO: 186 NAARDPARR, SEQ ID NO: 187 TPVRSGTARR, or SEQ ID NO: 188 WSRMDARR) were added. Reaction mixtures were incubated for 16 h at 37° C., prior to the direct addition of 10 ml to the T cell assay plates (see for example U.S. Pat. No. 5,589,458). ELISA was used to measure IFN-gamma T cell responses to individual HIP reaction mixtures. For each T cell clone, the ELISA absorbance values to all peptides were averaged and the standard deviation was calculated. If the absorbance value toward an individual HIP was three times the standard deviation (3×sd) above the average signal, the response toward the peptide was classified as positive.

Mass Spectrometric Analysis

Proteins in chromatographic fractions were reduced with OTT and digested with AspN. Resulting peptides were resolved by online chromatography on a C18 column and a 1200 Series HPLC system (Agilent Technologies). Analysis was carried out with a 6550 iFunnel Q-TOF LC/MS mass spectrometer. Prior to the analysis, mass/charge (m/z)-ratios of predicted ions (SEQ ID NO: 189 DLQTLALWSRM: 1333.6910, 667.3491, 445.2352; SEQ ID NO: 190 DLQTLALNAAR: 1185.6566, 593.3319, 395.8904) were added to a preferred ion list for targeted fragmentation. Manual inspection of fragmentation spectra was used to match the spectral ions to the predicted peptide fragmentation pattern.

All patents, patent applications, provisional applications, and publications referred to or cited herein are incorporated by reference in their entirety, including all figures and tables, to the extent they are not inconsistent with the explicit teachings of this specification.

While the disclosure is susceptible to various modifications and alternative forms, specific exemplary embodiments of the present invention have been shown by way of example in the drawings and have been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

What is claimed is:

1. An isolated hybrid insulin peptide comprising a first peptide having at least 90% sequence identity to at least one of SEQ ID NOs: 1-86, 191, 192, 196, and 221, covalently linked through a peptide bond to a second peptide having at least 90% sequence identity to at least one of SEQ ID NOs: 87-175, or a truncation thereof, wherein the first peptide is positioned N-terminal or C-terminal to the second peptide.

2. The isolated hybrid insulin peptide of claim 1, wherein the first peptide is identical to at least one of SEQ ID NOs: 1-86, 191, 192, 196, and 221 covalently linked through a peptide bond to the second peptide, the second peptide being identical to at least one of SEQ ID NOs: 87-175, or a truncation thereof.

3. The isolated hybrid insulin peptide of claim 1, wherein the first peptide is positioned N-terminal to the second peptide.

4. The isolated hybrid insulin peptide of claim 1, wherein the hybrid insulin peptide is formulated into a pharmaceutical composition.

5. The isolated hybrid insulin peptide of claim 1, wherein the human hybrid insulin peptide is antigenic for a diabetogenic CD4 T cell.

6. The isolated hybrid insulin peptide of claim 3, wherein the human hybrid insulin peptide is antigenic for a diabetogenic CD4 T cell.

7. The isolated hybrid insulin peptide of claim 1, wherein the first peptide comprises at least one amino acid sequence selected from the group consisting of
SEQ ID NO: 191, 192, 196, and 221.

8. A method for detecting a hybrid insulin peptide comprising performing an immunoassay or a T cell proliferation assay using the hybrid insulin peptide of claim 1.

9. The method for detecting a hybrid insulin peptide of claim 8, wherein the method comprises performing the immunoassay.

10. The method for detecting a hybrid insulin peptide of claim 9, wherein the immunoassay is an ELISA assay.

11. The method for detecting a hybrid insulin peptide of claim 10, wherein the ELISA assay is an ELISPOT assay.

12. The method for detecting a hybrid insulin peptide of claim 8, wherein the hybrid insulin peptide further comprises a hybrid insulin peptide-Major Histocompatability Complex multimer.

13. The method for detecting a hybrid insulin peptide of claim 8, wherein the method comprises performing the T cell proliferation assay.

14. A kit for detecting a hybrid insulin peptide comprising the isolated hybrid insulin peptide of claim 1.

15. The kit for detecting a hybrid insulin peptide of claim 14, wherein the kit further comprises at least on means for detecting the hybrid insulin peptide.

16. The kit for detecting a hybrid insulin peptide of claim 15, wherein the means for detecting the hybrid insulin peptide comprises an antibody and a detectable label.

17. The kit for detecting a hybrid insulin peptide of claim 16, wherein the detectable label is a fluorophore, an enzymatic label or a radiolabel.

18. The kit of claim 14, wherein the hybrid insulin peptide is conjugated to a solid support.


SEQUENCE LISTING (223 sequences)

SEQ ID NO: 1 - protein, 1 aa, Homo Sapiens
F

SEQ ID NO: 2 - protein, 2 aa, Homo Sapiens
FV

SEQ ID NO: 3 - protein, 3 aa, Homo Sapiens
FVN

SEQ ID NO: 4 - protein, 4 aa, Homo Sapiens
FVNQ

SEQ ID NO: 5 - protein, 5 aa, Homo Sapiens
FVNQH

SEQ ID NO: 6 - protein, 6 aa, Homo Sapiens
FVNQHL

SEQ ID NO: 7 - protein, 7 aa, Homo Sapiens
FVNQHLC

SEQ ID NO: 8 - protein, 8 aa, Homo Sapiens
FVNQHLCG

SEQ ID NO: 9 - protein, 9 aa, Homo Sapiens
FVNQHLCGS

SEQ ID NO: 10 - protein, 10 aa, Homo Sapiens
FVNQHLCGSH

SEQ ID NO: 11 - protein, 11 aa, Homo Sapiens
FVNQHLCGSHL

SEQ ID NO: 12 - protein, 12 aa, Homo Sapiens
FVNQHLCGSHLV

SEQ ID NO: 13 - protein, 13 aa, Homo Sapiens
FVNQHLCGSHLVE

SEQ ID NO: 14 - protein, 14 aa, Homo Sapiens
FVNQHLCGSHLVEA

SEQ ID NO: 15 - protein, 15 aa, Homo Sapiens
FVNQHLCGSHLVEAL

SEQ ID NO: 16 - protein, 16 aa, Homo Sapiens
FVNQHLCGSHLVEALY

SEQ ID NO: 17 - protein, 17 aa, Homo Sapiens
FVNQHLCGSHLVEALYL

SEQ ID NO: 18 - protein, 18 aa, Homo Sapiens
FVNQHLCGSHLVEALYLV

SEQ ID NO: 19 - protein, 19 aa, Homo Sapiens
FVNQHLCGSHLVEALYLVC

SEQ ID NO: 20 - protein, 20 aa, Homo Sapiens
FVNQHLCGSHLVEALYLVCG

SEQ ID NO: 21 - protein, 21 aa, Homo Sapiens
FVNQHLCGSHLVEALYLVCGE

SEQ ID NO: 22 - protein, 22 aa, Homo Sapiens
FVNQHLCGSHLVEALYLVCGER

SEQ ID NO: 23 - protein, 23 aa, Homo Sapiens
FVNQHLCGSHLVEALYLVCGERG

SEQ ID NO: 24 - protein, 24 aa, Homo Sapiens
FVNQHLCGSHLVEALYLVCGERGF

SEQ ID NO: 25 - protein, 25 aa, Homo Sapiens
FVNQHLCGSHLVEALYLVCGERGFF

SEQ ID NO: 26 - protein, 26 aa, Homo Sapiens
FVNQHLCGSHLVEALYLVCGERGFFY

SEQ ID NO: 27 - protein, 27 aa, Homo Sapiens
FVNQHLCGSHLVEALYLVCGERGFFYT

SEQ ID NO: 28 - protein, 28 aa, Homo Sapiens
FVNQHLCGSHLVEALYLVCGERGFFYTP

SEQ ID NO: 29 - protein, 29 aa, Homo Sapiens
FVNQHLCGSHLVEALYLVCGERGFFYTPK

SEQ ID NO: 30 - protein, 30 aa, Homo Sapiens
FVNQHLCGSHLVEALYLVCGERGFFYTPKT

SEQ ID NO: 31 - protein, 31 aa, Homo Sapiens
FVNQHLCGSHLVEALYLVCGERGFFYTPKTR

SEQ ID NO: 32 - protein, 32 aa, Homo Sapiens
FVNQHLCGSHLVEALYLVCGERGFFYTPKTRR

SEQ ID NO: 33 - protein, 33 aa, Homo Sapiens
FVNQHLCGSHLVEALYLVCGERGFFYTPKTRRE

SEQ ID NO: 34 - protein, 34 aa, Homo Sapiens
FVNQHLCGSHLVEALYLVCGERGFFYTPKTRREA

SEQ ID NO: 35 - protein, 35 aa, Homo Sapiens
FVNQHLCGSHLVEALYLVCGERGFFYTPKTRREAE

SEQ ID NO: 36 - protein, 36 aa, Homo Sapiens
FVNQHLCGSHLVEALYLVCGERGFFYTPKTRREAED

SEQ ID NO: 37 - protein, 37 aa, Homo Sapiens
FVNQHLCGSHLVEALYLVCGERGFFYTPKTRREAEDL

SEQ ID NO: 38 - protein, 38 aa, Homo Sapiens
FVNQHLCGSHLVEALYLVCGERGFFYTPKTRREAEDLQ

SEQ ID NO: 39 - protein, 39 aa, Homo Sapiens
FVNQHLCGSHLVEALYLVCGERGFFYTPKTRREAEDLQV

SEQ ID NO: 40 - protein, 40 aa, Homo Sapiens
FVNQHLCGSHLVEALYLVCGERGFFYTPKTRREAEDLQVG

SEQ ID NO: 41 - protein, 41 aa, Homo Sapiens
FVNQHLCGSHLVEALYLVCGERGFFYTPKTRREAEDLQVGQ

SEQ ID NO: 42 - protein, 42 aa, Homo Sapiens
FVNQHLCGSHLVEALYLVCGERGFFYTPKTRREAEDLQVGQV

SEQ ID NO: 43 - protein, 43 aa, Homo Sapiens
FVNQHLCGSHLVEALYLVCGERGFFYTPKTRREAEDLQVGQVE

SEQ ID NO: 44 - protein, 44 aa, Homo Sapiens
FVNQHLCGSHLVEALYLVCGERGFFYTPKTRREAEDLQVGQVEL

SEQ ID NO: 45 - protein, 45 aa, Homo Sapiens
FVNQHLCGSHLVEALYLVCGERGFFYTPKTRREAEDLQVGQVELG

SEQ ID NO: 46 - protein, 46 aa, Homo Sapiens
FVNQHLCGSHLVEALYLVCGERGFFYTPKTRREAEDLQVGQVELGG

SEQ ID NO: 47 - protein, 47 aa, Homo Sapiens
FVNQHLCGSHLVEALYLVCGERGFFYTPKTRREAEDLQVGQVELGGG

SEQ ID NO: 48 - protein, 48 aa, Homo Sapiens
FVNQHLCGSHLVEALYLVCGERGFFYTPKTRREAEDLQVGQVELGGGP

SEQ ID NO: 49 - protein, 49 aa, Homo Sapiens
FVNQHLCGSHLVEALYLVCGERGFFYTPKTRREAEDLQVGQVELGGGPG

SEQ ID NO: 50 - protein, 50 aa, Homo Sapiens
FVNQHLCGSHLVEALYLVCGERGFFYTPKTRREAEDLQVGQVELGGGPGA

SEQ ID NO: 51 - protein, 51 aa, Homo Sapiens
FVNQHLCGSHLVEALYLVCGERGFFYTPKTRREAEDLQVGQVELGGGPGAG

SEQ ID NO: 52 - protein, 52 aa, Homo Sapiens
FVNQHLCGSHLVEALYLVCGERGFFYTPKTRREAEDLQVGQVELGGGPGAGS

SEQ ID NO: 53 - protein, 53 aa, Homo Sapiens
FVNQHLCGSHLVEALYLVCGERGFFYTPKTRREAEDLQVGQVELGGGPGAGSL

SEQ ID NO: 54 - protein, 54 aa, Homo Sapiens
FVNQHLCGSHLVEALYLVCGERGFFYTPKTRREAEDLQVGQVELGGGPGAGSLQ

SEQ ID NO: 55 - protein, 55 aa, Homo Sapiens
FVNQHLCGSHLVEALYLVCGERGFFYTPKTRREAEDLQVGQVELGGGPGAGSLQP

SEQ ID NO: 56 - protein, 56 aa, Homo Sapiens
FVNQHLCGSHLVEALYLVCGERGFFYTPKTRREAEDLQVGQVELGGGPGAGSLQPL

SEQ ID NO: 57 - protein, 57 aa, Homo Sapiens
FVNQHLCGSHLVEALYLVCGERGFFYTPKTRREAEDLQVGQVELGGGPGAGSLQPLA

SEQ ID NO: 58 - protein, 58 aa, Homo Sapiens
FVNQHLCGSHLVEALYLVCGERGFFYTPKTRREAEDLQVGQVELGGGPGAGSLQPLAL

SEQ ID NO: 59 - protein, 59 aa, Homo Sapiens
FVNQHLCGSHLVEALYLVCGERGFFYTPKTRREAEDLQVGQVELGGGPGAGSLQPLALE

SEQ ID NO: 60 - protein, 60 aa, Homo Sapiens
FVNQHLCGSHLVEALYLVCGERGFFYTPKTRREAEDLQVGQVELGGGPGAGSLQPLALEG

SEQ ID NO: 61 - protein, 61 aa, Homo Sapiens
FVNQHLCGSHLVEALYLVCGERGFFYTPKTRREAEDLQVGQVELGGGPGAGSLQPLALEGS

SEQ ID NO: 62 - protein, 62 aa, Homo Sapiens
FVNQHLCGSHLVEALYLVCGERGFFYTPKTRREAEDLQVGQVELGGGPGAGSLQPLALEGSL

SEQ ID NO: 63 - protein, 63 aa, Homo Sapiens
FVNQHLCGSHLVEALYLVCGERGFFYTPKTRREAEDLQVGQVELGGGPGAGSLQPLALEGSLQ

SEQ ID NO: 64 - protein, 64 aa, Homo Sapiens
FVNQHLCGSHLVEALYLVCGERGFFYTPKTRREAEDLQVGQVELGGGPGAGSLQPLALEGSLQK

SEQ ID NO: 65 - protein, 65 aa, Homo Sapiens
FVNQHLCGSHLVEALYLVCGERGFFYTPKTRREAEDLQVGQVELGGGPGAGSLQPLALEGSLQKR

SEQ ID NO: 66 - protein, 66 aa, Homo Sapiens
FVNQHLCGSHLVEALYLVCGERGFFYTPKTRREAEDLQVGQVELGGGPGAGSLQPLALEGSLQKRG

SEQ ID NO: 67 - protein, 67 aa, Homo Sapiens
FVNQHLCGSHLVEALYLVCGERGFFYTPKTRREAEDLQVGQVELGGGPGAGSLQPLALEGSLQKRGI

SEQ ID NO: 68 - protein, 68 aa, Homo Sapiens
FVNQHLCGSHLVEALYLVCGERGFFYTPKTRREAEDLQVGQVELGGGPGAGSLQPLALEGSLQKRGIV

SEQ ID NO: 69 - protein, 69 aa, Homo Sapiens
FVNQHLCGSHLVEALYLVCGERGFFYTPKTRREAEDLQVGQVELGGGPGAGSLQPLALEGSLQKRGIVE

SEQ ID NO: 70 - protein, 70 aa, Homo Sapiens
FVNQHLCGSHLVEALYLVCGERGFFYTPKTRREAEDLQVGQVELGGGPGAGSLQPLALEGSLQKRGIVEQ

SEQ ID NO: 71 - protein, 71 aa, Homo Sapiens
FVNQHLCGSHLVEALYLVCGERGFFYTPKTRREAEDLQVGQVELGGGPGAGSLQPLALEGSLQKRGIVEQC

SEQ ID NO: 72 - protein, 72 aa, Homo Sapiens
FVNQHLCGSHLVEALYLVCGERGFFYTPKTRREAEDLQVGQVELGGGPGAGSLQPLALEGSLQKRGIVEQCC

SEQ ID NO: 73 - protein, 73 aa, Homo Sapiens
FVNQHLCGSHLVEALYLVCGERGFFYTPKTRREAEDLQVGQVELGGGPGAGSLQPLALEGSLQKRGIVEQCCT

SEQ ID NO: 74 - protein, 74 aa, Homo Sapiens
FVNQHLCGSHLVEALYLVCGERGFFYTPKTRREAEDLQVGQVELGGGPGAGSLQPLALEGSLQKRGIVEQCCTS

SEQ ID NO: 75 - protein, 75 aa, Homo Sapiens
FVNQHLCGSHLVEALYLVCGERGFFYTPKTRREAEDLQVGQVELGGGPGAGSLQPLALEGSLQKRGIVEQCCTSI

SEQ ID NO: 76 - protein, 76 aa, Homo Sapiens
FVNQHLCGSHLVEALYLVCGERGFFYTPKTRREAEDLQVGQVELGGGPGAGSLQPLALEGSLQKRGIVEQCCTSIC

SEQ ID NO: 77 - protein, 77 aa, Homo Sapiens
FVNQHLCGSHLVEALYLVCGERGFFYTPKTRREAEDLQVGQVELGGGPGAGSLQPLALEGSLQKRGIVEQCCTSICS

SEQ ID NO: 78 - protein, 78 aa, Homo Sapiens
FVNQHLCGSHLVEALYLVCGERGFFYTPKTRREAEDLQVGQVELGGGPGAGSLQPLALEGSLQKRGIVEQCCTSICSL

SEQ ID NO: 79 - protein, 79 aa, Homo Sapiens
FVNQHLCGSHLVEALYLVCGERGFFYTPKTRREAEDLQVGQVELGGGPGAGSLQPLALEGSLQKRGIVEQCCTSICSLY

SEQ ID NO: 80 - protein, 80 aa, Homo Sapiens
FVNQHLCGSHLVEALYLVCGERGFFYTPKTRREAEDLQVGQVELGGGPGAGSLQPLALEGSLQKRGIVEQCCTSICSLYQ

SEQ ID NO: 81 - protein, 81 aa, Homo Sapiens
FVNQHLCGSHLVEALYLVCGERGFFYTPKTRREAEDLQVGQVELGGGPGAGSLQPLALEGSLQKRGIVEQCCTSICSLYQL

SEQ ID NO: 82 - protein, 82 aa, Homo Sapiens
FVNQHLCGSHLVEALYLVCGERGFFYTPKTRREAEDLQVGQVELGGGPGAGSLQPLALEGSLQKRGIVEQCCTSICSLYQLE

SEQ ID NO: 83 - protein, 83 aa, Homo Sapiens
FVNQHLCGSHLVEALYLVCGERGFFYTPKTRREAEDLQVGQVELGGGPGAGSLQPLALEGSLQKRGIVEQCCTSICSLYQLEN

SEQ ID NO: 84 - protein, 84 aa, Homo Sapiens
FVNQHLCGSHLVEALYLVCGERGFFYTPKTRREAEDLQVGQVELGGGPGAGSLQPLALEGSLQKRGIVEQCCTSICSLYQLENY

SEQ ID NO: 85 - protein, 85 aa, Homo Sapiens
FVNQHLCGSHLVEALYLVCGERGFFYTPKTRREAEDLQVGQVELGGGPGAGSLQPLALEGSLQKRGIVEQCCTSICSLYQLENYC

SEQ ID NO: 86 - protein, 86 aa, Homo Sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 87 - protein, 30 aa, Homo Sapiens; identical to SEQ ID NO: 30

SEQ ID NO: 88 - protein, 21 aa, Homo Sapiens
GIVEQCCTSICSLYQLENYCN

SEQ ID NO: 89 - protein, 31 aa, Homo Sapiens
EAEDLQVGQVELGGGPGAGSLQPLALEGSLQ

SEQ ID NO: 90 - protein, 131 aa, Homo Sapiens
ASFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPE

SEQ ID NO: 91 - protein, 19 aa, Homo Sapiens
LKHMQFPPMYEENSRDNPF

SEQ ID NO: 92 - protein, 33 aa, Homo Sapiens
TNEIVEEQYTPQSLATLESVFQELGKLTGPNNQ

SEQ ID NO: 93 - protein, 66 aa, Homo Sapiens
ERMDEEQKLYTDDEDDIYKANNIAYEDVVGGEDWNPVEEKIESQTQEEVRDSKENIEKNEQINDEM

SEQ ID NO: 94 - protein, 11 aa, Homo Sapiens
SGQLGIQEEDL

SEQ ID NO: 95 - protein, 17 aa, Homo Sapiens
ESKDQLSDDVSKVIAYL

SEQ ID NO: 96 - protein, 44 aa, Homo Sapiens
LVNAAGSGRLQNGQNGERATRLFEKPLDSQSIYQLIEISRNLQI

SEQ ID NO: 97 - protein, 40 aa, Homo Sapiens
VPGQGSSEDDLQEEEQIEQAIKEHLNQGSSQETDKLAPVS

SEQ ID NO: 98 - protein, 42 aa, Homo Sapiens
FPVGPPKNDDTPNRQYWDEDLLMKVLEYLNQEKAEKGREHIA

SEQ ID NO: 99 - protein, 5 aa, Homo Sapiens
AMENM

SEQ ID NO: 100 - protein, 76 aa, Homo Sapiens
LPVNSPMNKGDTEVMKCIVEVISDTLSKPSPMPVSQECFETLRGDERILSILRHQNLLKELQDLALQGAKERAHQQ

SEQ ID NO: 101 - protein, 35 aa, Homo Sapiens
HSGFEDELSEVLENQSSQAELKEAVEEPSSKDVME

SEQ ID NO: 102 - protein, 92 aa, Homo Sapiens
EDSKEAEKSGEATDGARPQALPEPMQESKAEGNNQAPGEEEEEEEEATNTHPPASLPSQKYPGPQAEGDSEGLSQGLVDREKGLSAEPGWQA

SEQ ID NO: 103 - protein, 38 aa, Homo Sapiens
EEEEEEEEEAEAGEEAVPEEEGPTVVLNPHPSLGYKEI

SEQ ID NO: 104 - protein, 72 aa, Homo Sapiens
GESRSEALAVDGAGKPGAEEAQDPEGKGEQEHSQQKEEEEEMAVVPQGLFRGGKSGELEQEEERLSKEWEDS

SEQ ID NO: 105 - protein, 94 aa, Homo Sapiens
MRSAAVLALLLCAGQVTALPVNSPMNKGDTEVMKCIVEVISDTLSKPSPMPVSQECFETLRGDERILSILRHQNLLKELQDLALQGAKERAHQQ

SEQ ID NO: 106 - protein, 14 aa, Homo Sapiens
WSKMDQLAKELTAE

SEQ ID NO: 107 - protein, 33 aa, Homo Sapiens
LEGQEEEEDNRDSSMKLSFRARAYGFRGPGPQL

SEQ ID NO: 108 - protein, 25 aa, Homo Sapiens
GWRPSSREDSLEAGLPLQVRGYPEE

SEQ ID NO: 109 - protein, 7 aa, Homo Sapiens
EEEGSAN

SEQ ID NO: 110 - protein, 26 aa, Homo Sapiens
PEDQELESLSAIEAELEKVAHQLQAL

SEQ ID NO: 111 - protein, 44 aa, Homo Sapiens
MPVDNRNHNEGMVTRCIIEVLSNALSKSSAPPITPECRQVLKTS

SEQ ID NO: 112 - protein, 135 aa, Homo Sapiens
DVKDKETTENENTKFEVRLLRDPADASEAHESSSRGEAGAPGEEDIQGPTKADTEKWAEGGGHSRERADEPQWSLYPSDSQVSEEVKTRHSEKSQREDEEEEEGENYQKGERGEDSSEEKHLEEPGETQNAFLNE

SEQ ID NO: 113 - protein, 5 aa, Homo Sapiens
QASAI

SEQ ID NO: 114 - protein, 65 aa, Homo Sapiens
EELVARSETHAAGHSQEKTHSREKSSQESGEETGSQENHPQESKGQPRSQEESEEGEEDATSEVD

SEQ ID NO: 115 - protein, 46 aa, Homo Sapiens
RTRPRHHHGRSRPDRSSQGGSLPSEEKGHPQEESEESNVSMASLGE

SEQ ID NO: 116 - protein, 60 aa, Homo Sapiens
DHHSTHYRASEEEPEYGEEIKGYPGVQAPEDLEWERYRGRGSEEYRAPRPQSEESWDEED

SEQ ID NO: 117 - protein, 50 aa, Homo Sapiens
NYPSLELDKMAHGYGEESEEERGLEPGKGRHHRGRGGEPRAYFMSDTREE

SEQ ID NO: 118 - protein, 17 aa, Homo Sapiens
FLGEGHHRVQENQMDKA

SEQ ID NO: 119 - protein, 55 aa, Homo Sapiens
HPQGAWKELDRNYLNYGEEGAPGKWQQQGDLQDTKENREEARFQDKQYSSHHTAE

SEQ ID NO: 120 - protein, 20 aa, Homo Sapiens
LGELFNPYYDPLQWKSSHFE

SEQ ID NO: 121 - protein, 33 aa, Homo Sapiens
DNMNDNFLEGEEENELTLNEKNFFPEYNYDWWE

SEQ ID NO: 122 - protein, 11 aa, Homo Sapiens
PFSEDVNWGYE

SEQ ID NO: 123 - protein, 10 aa, Homo Sapiens
NLARVPKLDL

SEQ ID NO: 124 - protein, 14 aa, Homo Sapiens
QYDRVAQLDQLLHY

SEQ ID NO: 125 - protein, 37 aa, Homo Sapiens
KSAEFPDFYDSEEPVSTHQEAENEKDRADQTVLTEDE

SEQ ID NO: 126 - protein, 23 aa, Homo Sapiens
ELENLAAMDLELQKIAEKFSQRG

SEQ ID NO: 127 - protein, 23 aa, Homo Sapiens
ARPVKEPRGLSAASPPLAETGAP

SEQ ID NO: 128 - protein, 154 aa, Homo Sapiens
SVPRGEAAGAVQELARALAHLLEAERQERARAEAQEAEDQQARVLAQLLRVWGAPRNSDPALGLDDDPDAPAAQLARALLRARLDPAALAAQLVPAPVPAAALRPRPPVYDDGPAGPDAEEAGDETPDVDPELLRYLLGRILAGSADSEGVAAP

SEQ ID NO: 129 - protein, 22 aa, Homo Sapiens
AADHDVGSELPPEGVLGALLRV

SEQ ID NO: 130 - protein, 10 aa, Homo Sapiens
LETPAPQVPA

SEQ ID NO: 131 - protein, 4 aa, Homo Sapiens
LLPP

SEQ ID NO: 132 - protein, 32 aa, Homo Sapiens
ERPVFTNHFLVELHKGGEDKARQVAAEHGFGV

SEQ ID NO: 133 - protein, 16 aa, Homo Sapiens
LPFAEGLYHFYHNGLA

SEQ ID NO: 134 - protein, 24 aa, Homo Sapiens
SLHHKQQLERDPRVKMALQQEGFD

SEQ ID NO: 135 - protein, 250 aa, Homo Sapiens
GYRDINEIDINMNDPLFTKQWYLINTGQADGTPGLDLNVAEAWELGYTGKGVTIGIMDDGIDYLHPDLASNYNAEASYDFSSNDPYPYPRYTDDWFNSHGTRCAGEVSAAANNNICGVGVAYNSKVAGIRMLDQPFMTDIIEASSISHMPQLIDIYSASWGPTDNGKTVDGPRELTLQAMADGVNKGRGGKGSIYVWASGDGGSYDDCNCDGYASSMWTISINSAINDGRTALYDESCSSTLASTFSNGR

SEQ ID NO: 136 - protein, 56 aa, Homo Sapiens
NPEAGVATTDLYGNCTLRHSGTSAAAPEAAGVFALALEANLGLTWRDMQHLTVLTS

SEQ ID NO: 137 - protein, 10 aa, Homo Sapiens
NQLHDEVHQW

SEQ ID NO: 138 - protein, 85 aa, Homo Sapiens
NGVGLEFNHLFGYGVLDAGAMVKMAKDWKTVPERFHCVGGSVQDPEKIPSTGKLVLTLTTDACEGKENFVRYLEHVQAVITVNAT

SEQ ID NO: 139 - protein, 19 aa, Homo Sapiens
GDLNINMTSPMGTKSILLS

SEQ ID NO: 140 - protein, 75 aa, Homo Sapiens
PRDDDSKVGFDKWPFMTTHTWGEDARGTWTLELGFVGSAPQKGVLKEWTLMLHGTQSAPYIDQVVRDYQSKLAMS

SEQ ID NO: 141 - protein, 22 aa, Homo Sapiens
EELEEELDEAVERSLKSILNKN

SEQ ID NO: 142 - protein, 4 aa, Homo Sapiens
EEED

SEQ ID NO: 143 - protein, 71 aa, Homo Sapiens
EDVGTVVGIDLGTTYSCVGVFKNGRVEIIANDQGNRITPSYVAFTPEGERLIGDAAKNQLTSNPENTVFDA

SEQ ID NO: 144 - protein, 24 aa, Homo Sapiens
LIGRTWNDPSVQQDIKFLPFKVVE

SEQ ID NO: 145 - protein, 39 aa, Homo Sapiens
TKPYIQVDIGGGQTKTFAPEEISAMVLTKMKETAEAYLG

SEQ ID NO: 146 - protein, 48 aa, Homo Sapiens
VTHAVVTVPAYFNDAQRQATKDAGTIAGLNVMRIINEPTAAAIAYGLD

SEQ ID NO: 147 - protein, 56 aa, Homo Sapiens
EGEKNILVFDLGGGTFDVSLLTIDNGVFEVVATNGDTHLGGEDFDQRVMEHFIKLY

SEQ ID NO: 148 - protein, 6 aa, Homo Sapiens
KTGKDV

SEQ ID NO: 149 - protein, 8 aa, Homo Sapiens
DNRAVQKL

SEQ ID NO: 150 - protein, 5 aa, Homo Sapiens
EVEKA

SEQ ID NO: 151 - protein, 54 aa, Homo Sapiens
ALSSQHQARIEIESFYEGEDFSETLTRAKFEELNMDLFRSTMKPVQKVLEDSDL

SEQ ID NO: 152 - protein, 92 aa, Homo Sapiens
SDIDEIVLVGGSTRIPKIQQLVKEFFNGKEPSRGINPDEAVAYGAAVQAGVLSGDQDTGDLVLLDVCPLTLGIETVGGVMTKLIPRNTVVPT

SEQ ID NO: 153 - protein, 105 aa, Homo Sapiens
SQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEED

SEQ ID NO: 154 - protein, 64 aa, Homo Sapiens
LKERIDTRNELESYAYSLKNQIGDKEKLGGKLSSEDKETMEKAVEEKIEWLESHQDADIEDFKA

SEQ ID NO: 155 - protein, 34 aa, Homo Sapiens
KELEEIVQPIISKLYGSAGPPPTGEEDTAEKDEL

SEQ ID NO: 156 - protein, 17 aa, Homo Sapiens
RSVNPYLQGQRLDNVVA

SEQ ID NO: 157 - protein, 13 aa, Homo Sapiens
SVPHFSDEDKDPE

SEQ ID NO: 158 - protein, 36 aa, Homo Sapiens
YPSKPDNPGEDAPAEDMARYYSALRHYINLITRQRY

SEQ ID NO: 159 - protein, 30 aa, Homo Sapiens
SSPETLISDLLMRESTENVPRTRLEDPAMW

SEQ ID NO: 160 - protein, 35 aa, Homo Sapiens
FPKPGGSQDKSLHNRELSAERPLNEQIAEAEEDKI

SEQ ID NO: 161 - protein, 57 aa, Homo Sapiens
TYPPENKPGQSNYSFVDNLNLLKAITEKEKIEKERQSIRSSPLDNKLNVEDVDSTKN

SEQ ID NO: 162 - protein, 145 aa, Homo Sapiens
LIDDYDSTKSGLDHKFQDDPDGLHQLDGTPLTAEDIVHKIAARIYEENDRAVFDKIVSKLLNLGLITESQAHTLEDEVAEVLQKLISKEANNYEEDPNKPTSWTENQAGKIPEKVTPMAAIQDGLAKGENDETVSNTLTLTNGLE

SEQ ID NO: 163 - protein, 150 aa, Homo Sapiens
TKTYSEDNFEELQYFPNFYALLKSIDSEKEAKEKETLITIMKTLIDFVKMMVKYGTISPEEGVSYLENLDEMIALQTKNKLEKNATDNISKLFPAPSEKSHEETDSTKEEAAKMEKEYGSLKDSTKDDNSNPGGKTDEPKGKTEAYLEAI

SEQ ID NO: 164 - protein, 5 aa, Homo Sapiens
NIEWL

SEQ ID NO: 165 - protein, 2 aa, Homo Sapiens
HD

SEQ ID NO: 166 - protein, 36 aa, Homo Sapiens
GNKEDYDLSKMRDFINKQADAYVEKGILDKEEAEAI

SEQ ID NO: 167 - protein, 5 aa, Homo Sapiens
IYSSL

SEQ ID NO: 168 - protein, 11 aa, Homo Sapiens
TPIESHQVEKR

SEQ ID NO: 169 - protein, 38 aa, Homo Sapiens
KCNTATCATQRLANFLVHSSNNFGAILSSTNVGSNTYG

SEQ ID NO: 170 - protein, 16 aa, Homo Sapiens
NAVEVLKREPLNYLPL

SEQ ID NO: 171 - protein, 36 aa, Homo Sapiens
AYRPSETLCGGELVDTLQFVCGDRGFYFSRPASRVS

SEQ ID NO: 172 - protein, 64 aa, Homo Sapiens
SRGIVEECCFRSCDLALLETYCATPAKSERDVSTPPTVLPDNFPRYPVGKFFQYDTWKQSTQRL

SEQ ID NO: 173 - protein, 8 aa, Homo Sapiens
GLPALLRA

SEQ ID NO: 174 - protein, 14 aa, Homo Sapiens
GHVLAKELEAFREA

SEQ ID NO: 175 - protein, 24 aa, Homo Sapiens
HRPLIALPTQDPAHGGAPPEMASN

SEQ ID NO: 176 - protein, 27 aa, Homo Sapiens
FVNQHLCGSHLVEGHVLAKELEAFREA

SEQ ID NO: 177 - protein, 9 aa, Artificial Sequence
  synthetic polypeptide
RRAHLVEAL

SEQ ID NO: 178 - protein, 9 aa, Artificial Sequence
  synthetic polypeptide
RRALVEALY

SEQ ID NO: 179 - protein, 9 aa, Artificial Sequence
  synthetic polypeptide
RRAVEALYL

SEQ ID NO: 180 - protein, 9 aa, Artificial Sequence
  synthetic polypeptide
RRAGDLQTL

SEQ ID NO: 181 - protein, 9 aa, Artificial Sequence
  synthetic polypeptide
RRADLQTLA

SEQ ID NO: 182 - protein, 9 aa, Artificial Sequence
  synthetic polypeptide
RRALQTLAL

SEQ ID NO: 183 - protein, 9 aa, Artificial Sequence
  synthetic polypeptide
RRAQTLALE

SEQ ID NO: 184 - protein, 9 aa, Artificial Sequence
  synthetic polypeptide
RRATLALEV

SEQ ID NO: 185 - protein, 9 aa, Artificial Sequence
  synthetic polypeptide
KCNTATARR

SEQ ID NO: 186 - protein, 9 aa, Artificial Sequence
  synthetic polypeptide
NAARDPARR

SEQ ID NO: 187 - protein, 10 aa, Artificial Sequence
  synthetic polypeptide
TPVRSGTARR

SEQ ID NO: 188 - protein, 8 aa, Artificial Sequence
  synthetic polypeptide
WSRMDARR

SEQ ID NO: 189 - protein, 11 aa, Artificial Sequence
  synthetic polypeptide
DLQTLALWSRM

SEQ ID NO: 190 - protein, 11 aa, Artificial Sequence
  synthetic polypeptide
DLQTLALNAAR

SEQ ID NO: 191 - protein, 11 aa, Mus Musculus
DPQVAQLELGG

SEQ ID NO: 192 - protein, 7 aa, Mus Musculus
DLQTLAL

SEQ ID NO: 193 - protein, 12 aa, Mus Musculus
EVEDPQVAQLEL

SEQ ID NO: 194 - protein, 23 aa, Artificial Sequence
  synthetic polypeptide
DPQVAQLELGGEVEDPQVAQLEL

SEQ ID NO: 195 - protein, 13 aa, Artificial Sequence
  synthetic polypeptide
DLQTLALEVEDPQ

SEQ ID NO: 196 - protein, 6 aa, Mus Musculus
LQTLAL

SEQ ID NO: 197 - protein, 11 aa, Artificial Sequence
  synthetic polypeptide
LQTLALNAARD

SEQ ID NO: 198 - protein, 5 aa, Mus Musculus
NAARD

SEQ ID NO: 199 - protein, 11 aa, Artificial Sequence
  synthetic polypeptide
LQTLALWSRMD

SEQ ID NO: 200 - protein, 6 aa, Mus Musculus
GDLQTL

SEQ ID NO: 201 - protein, 6 aa, Mus Musculus
DLQTLA

SEQ ID NO: 202 - protein, 6 aa, Mus Musculus
QTLALE

SEQ ID NO: 203 - protein, 6 aa, Mus Musculus
TLALEV

SEQ ID NO: 204 - protein, 6 aa, Mus Musculus
HLVEAL

SEQ ID NO: 205 - protein, 6 aa, Mus Musculus
LVEALY

SEQ ID NO: 206 - protein, 6 aa, Mus Musculus
VEALYL

SEQ ID NO: 207 - protein, 5 aa, Mus Musculus
WSRMD

SEQ ID NO: 208 - protein, 5 aa, Mus Musculus
TPVRS

SEQ ID NO: 209 - protein, 5 aa, Mus Musculus
KCNTA

SEQ ID NO: 210 - protein, 5 aa, Mus Musculus
NAARD

SEQ ID NO: 211 - protein, 4 aa, Mus Musculus
WSRM

SEQ ID NO: 212 - protein, 19 aa, Mus Musculus
EVEDPQVAQLELGGGPGAG

SEQ ID NO: 213 - protein, 20 aa, Mus Musculus
EVEDPQVAQLELGGGPGAGD

SEQ ID NO: 214 - protein, 21 aa, Mus Musculus
EVEDPQVAQLELGGGPGAGDL

SEQ ID NO: 215 - protein, 22 aa, Mus Musculus
EVEDPQVAQLELGGGPGAGDLQ

SEQ ID NO: 216 - protein, 23 aa, Mus Musculus
EVEDPQVAQLELGGGPGAGDLQT

SEQ ID NO: 217 - protein, 24 aa, Mus Musculus
EVEDPQVAQLELGGGPGAGDLQTL

SEQ ID NO: 218 - protein, 25 aa, Mus Musculus
EVEDPQVAQLELGGGPGAGDLQTLA

SEQ ID NO: 219 - protein, 26 aa, Mus Musculus
EVEDPQVAQLELGGGPGAGDLQTLAL

SEQ ID NO: 220 - protein, 31 aa, Mus Musculus
EVEDPQVAQLELGGGPGAGDLQTLALEVAQQ

SEQ ID NO: 221 - protein, 6 aa, Mus Musculus
DQTLAL

SEQ ID NO: 222 - protein, 138 aa, Homo Sapiens
YSPRTPDRVSEADIQRLLHGVMEQLGIARPRVEYPAHQAMNLVGPQSIEGGAHEGLQHLGPFGNIPNIVAELTGDNIPKDFSEDQGYPDPPNPCPVGKTADDGCLENTPDTAEFSREFQLHQHLFDPEHDYPGLGKWN

SEQ ID NO: 223 - protein, 10 aa, Homo Sapiens
LLYEKMKGGE